(19) United States
(12) Patent Application Publication
(10) Pub. No.: US 2009/0012116 A1
(43) Pub. Date: Jan. 8, 2009

(54) Muscarinic Receptor Antagonists

(76) Inventors: Naresh Kumar, Gurgaon (IN); Kirandeep Kaur, Gurgaon (IN); Sandeep Sinha, New Delhi (IN); Suman Gupta, Gurgaon (IN); Venkata P. Palle, Ganeshkhind (IN); Anita Chugh, New Delhi (IN)

Correspondence Address: RANBAXY INC., 600 COLLEGE ROAD EAST, SUITE 2100, PRINCETON, NJ, 08540, US

(21) Appl. No.: 11/995,376

(22) PCT Filed: Jul. 11, 2006
(86) PCT No.: PCT/IB06/52350

§ 371 Date: Sep. 10, 2008

(30) Foreign Application Priority Data
Jul. 11, 2005 (IN) 1797/DEL/2005

(51) Int. Cl. A61K 31/44; C07D 453/02; A61K 31/40; C07D 487/08

(52) U.S. Cl. 514/305; 546/133; 548/453; 514/413

(57) ABSTRACT

Provided are muscarinic receptor antagonists, which can be useful in treating various diseases of the respiratory, urinary or gastrointestinal system mediated through muscarinic receptors. Also provided are processes for preparing compounds described herein, pharmaceutical compositions comprising compounds described herein, and methods for treating diseases mediated through muscarinic receptors.

### FIELD OF THE INVENTION

Provided are muscarinic receptor antagonists, which can be useful in treating various diseases of the respiratory, urinary or gastrointestinal system mediated through muscarinic receptors. Also provided are processes for preparing compounds described herein, pharmaceutical compositions comprising compounds described herein, and methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented (for example, the M₁ subtype is located primarily in neuronal tissues such as cerebral cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

Biological potentials of modulating muscarinic receptor subtypes by ligands in different disease conditions, such as Alzheimer's disease, pain, urinary disease condition, chronic obstructive pulmonary disease, and the like, have been disclosed. (Curr. Opin. Chem. Biol., 3, p. 426 (1999), Trends in Pharmacol. Sci., 22, p. 409 (2001)). The pharmacological and medical aspects of the muscarinic class of acetylcholine agonists and antagonists have been disclosed. (Molecules, 6, p. 142 (2001)). Recent developments on the role of different muscarinic receptor subtypes using different muscarinic receptor of knock out mice have been disclosed. (Trends in Pharmacol. Sci., 22, p. 215 (2001)).

Most smooth muscle express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there is a need for novel muscarinic receptor antagonists that demonstrate target organ selectivity.

WO 04/005252 discloses azabicyclo derivatives described as musacrinic receptor antagonists. WO 04/004629, WO 04/052857, WO 04/067510, WO 04/014853, WO 04/014363 discloses 3,6-disubstituted azabicyclo[3.1.0]hexane derivatives described as useful muscarinic receptor antagonists. WO2004/056811 discloses flaxavate derivatives as muscarinic receptor antagonists. WO2004/056810 discloses xanthene derivatives as muscarinic receptor antagonists. WO2004/056767 discloses 1-substituted-3-pyrrolidine derivatives as muscarinic receptor antagonists. WO2004/089363, WO2004/089898, WO04069835, WO2004/089900 and WO2004089364 discloses substituted azabicyclohexane derivatives as muscarinic receptor antagonists. WO2006/018708 disclose pyrrolidine derivatives as muscarinic receptor antagonists. WO2006/35303 discloses azabicyclo derivatives as muscarinic receptor antagonists.

Cyclohexylmethylpiperidinyl-triphenylpropioamide derivatives as selective M₃ antagonist discriminating against the other receptor subtypes have been disclosed. (J. Med. Chem., 44, p. 984 (2002)). The synthesis and antimuscarinic activity of some 1-cycloalkyl-1-hydroxy-1-phenyl-3-(4-substituted piperazinyl)-2-propanones and related compounds have been disclosed. (J. Med. Chem., 36, p. 610 (1993)). Analogues of oxybutynin, synthesis and antimuscarinic activity of some substituted 7-amino-1-hydroxy-5-heptyn-2-ones and related compounds have been described. (J. Med. Chem., 34, p. 3065 (1991)). The synthesis and activity of analogues of Oxybutynin and Tolterodine. (Bio-Organic Medicinal Chemistry Letters, 15, p. 2093 (2005)).

In view of the above, however, there remains a need for muscarinic receptor antagonists useful in treating disease states associated with improper smooth muscle function and respiratory disorders.

### SUMMARY OF THE INVENTION

Generally provided are muscarinic receptor antagonists, which can be useful as safe and effective therapeutic or prophylactic agents for the treatment of various diseases of the respiratory, urinary or gastrointestinal system. Also provided are processes for synthesizing such compounds described herein.

Pharmaceutical compositions containing such compounds are also generally provided together with acceptable carriers, excipients or diluents. Such pharmaceutical compositions can be useful for the treatment of various diseases of the respiratory, urinary or gastrointestinal system.

Enantiomers, diastereomers, N-oxides, polymorphs, pharmaceutically acceptable salts and pharmaceutically acceptable solvates of the compounds described herein, as well as metabolites having the same type of activity are also provided, as well as pharmaceutical compositions comprising the compounds described herein, their metabolites, enantiomers, diastereomers, N-oxides, polymorphs, solvates or pharmaceutically acceptable salts thereof, in combination with one or more pharmaceutically acceptable carriers and one or more optional excipients.

Thus in one aspect, provided are compounds of Formula I

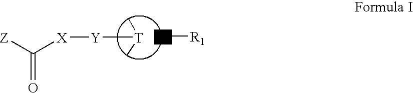

and pharmaceutically accepted salts, pharmaceutically acceptable solvates, enantiomers, diastereomers, polymorphs or N-oxides thereof, wherein

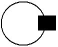

represents a nitrogen containing cyclic ring have 4-8 carbons;
T can be a bridging group selected from —(CH₂)ₙ—, —CH(Q)CH₂—, —CH₂CH(Q)CH₂—, —CH(Q)-, —CH₂—O—CH₂— or —CH₂—NH—CH₂—,

wherein

the bridging group can be attached to two carbon atoms of the ring

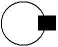

Q can be alkyl, alkenyl, alkynyl, cycloalkyl, aryl, heteroaryl, heterocyclyl, aralkyl, heterocyclylalkyl or heteroarylalkyl; and
n can be an integer selected from 0-3 (wherein when n is zero then T represents a direct bond);
X can be O, S or NRₛ,

wherein
Rₛ can be selected from hydrogen, alkyl, cycloalkyl, aryl, heteroaryl, heterocyclyl, heteroaryl, aralkyl, heteroarylalkyl or heterocyclylalkyl;
Y can be alkylene or no atom,

wherein when Y can be no atom then X is directly attached to the ring

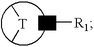

Z can be —NHR₂, —N(R₂)₂, aryl or cycloalkyl,

wherein
R₂ can be independently selected from alkyl, aryl, aralkyl, heteroaryl, cycloalkyl, heterocyclyl, heterocyclylalkyl or heteroarylalkyl; and
R₁ can be selected from hydrogen, aralkyl or Rᵤ,

wherein
Rᵤ can be alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
   wherein
   R₃ can be alkyl, aryl, heteroaryl, heterocyclyl, cycloalkyl, aralkyl, heteroarylalkyl, heterocyclylalkyl or —NRₓR_{y}, and
   Rₓ and R_{y} can independently be selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring.

In another aspect, provided are compounds selected from:
(3-Benzyl-3-azabicyclo[3.1.0]hex-6-yl)methyl biphenyl-2-ylcarbamate (Compound No. 1),
N-[(3-benzyl-3-azabicyclo[3.1.0]hex-6-yl)methyl]-N′-biphenyl-2-ylurea (Compound No. 2),
Tartarate salt of 3-azabicyclo[3.1.0]hex-6-ylmethyl biphenyl-2-ylcarbamate (Compound No. 3),
3-Benzyl-3-azabicyclo[3.2.1]oct-8-yl biphenyl-2-ylcarbamate (Compound No. 4),
3-azabicyclo[3.2.1]oct-8-yl biphenyl-2-ylcarbamate (Compound No. 5),
2-Benzyl-2-azabicyclo[2.2.1]hept-7-yl biphenyl-2-ylcarbamate (Compound No. 6),
(3-Benzyl-3-azabicyclo[3.2.1]oct-8-yl)methyl biphenyl-2-ylcarbamate (Compound No. 7),
(3-Benzyl-3-azabicyclo[3.1.0]hex-1-yl)methyl biphenyl-2-ylcarbamate (Compound No. 8),
(3-Benzyl-3-azabicyclo[3.1.0]hex-6-yl)methyl[2-(2-thienyl)phenyl]carbamate (Compound No. 9),
3-azabicyclo[3.1.0]hex-6-ylmethyl[2-(2-thienyl)phenyl]carbamate (Compound No. 10),
(3-Benzyl-3-azabicyclo[3.1.0]hex-6-yl)methyl(2′,4′-difluorobiphenyl-2-yl)carbamate (Compound No. 11),
(3-Benzyl-3-azabicyclo[3.1.0]hex-1-yl)methyl(2′,4′-difluorobiphenyl-2-yl)carbamate (Compound No. 12),
(3-Benzyl-3-azabicyclo[3.1.0]hex-6-yl)methyl(2′,4′-dimethoxybiphenyl-2-yl)carbamate (Compound No. 13),
3-Azabicyclo[3.1.0]hex-6-ylmethyl (2-fluorobenzyl)phenylcarbamate (Compound No. 14),
3-Azabicyclo[3.1.0]hex-6-ylmethyl benzyl(phenyl)carbamate (Compound No. 15),
(3-Benzyl-3-azabicyclo[3.1.0]hex-6-yl)methyl benzyl(3-fluorophenyl)carbamate (Compound No. 16),
3-Azabicyclo[3.1.0]hex-6-ylmethyl (2′,4′-difluorobiphenyl-2-yl)carbamate (Compound No. 17),
3-Azabicyclo[3.1.0]hex-6-ylmethyl (2′,4′-dimethoxybiphenyl-2-yl)carbamate (Compound No. 18),
3-Benzyl-3-azabicyclo[3.1.0]hex-6-yl)methyl[2-(1,3-benzodioxol-5-yl)phenyl]carbamate (Compound No. 19),
(3-Benzyl-3-azabicyclo[3.2.1]oct-8-yl)methyl(2-benzylphenyl)carbamate (Compound No. 20),
N-[(3-benzyl-3-azabicyclo[3.2.1]oct-8-yl)methyl]biphenyl-2-carboxamide (Compound No. 21),
3-Azabicyclo[3.1.0]hex-6-ylmethyl (2-benzylphenyl)carbamate (Compound No. 22),
2-Benzyl-N-[(3-benzyl-3-azabicyclo[3.2.1]oct-8-yl)methyl]benzamide (Compound No. 23),
(3-Benzyl-3-azabicyclo[3.2.1]oct-8-yl)methyl benzyl (4-fluorophenyl)carbamate. (Compound No. 24),
(3-Benzyl-3-azabicyclo[3.2.1]oct-8-yl)methyl(2-fluorobenzyl)phenyl carbamate. (Compound No. 25),
(3-Benzyl-3-azabicyclo[3.2.1]oct-8-yl)methyl benzyl(phenyl)carbamate (Compound No. 26),
(3-Benzyl-3-azabicyclo[3.2.1]oct-8-yl)methyl(4-methylbenzyl)phenyl carbamate (Compound No. 27),
(3-benzyl-3-azabicyclo[3.2.1]oct-8-yl)methyl(2-benzoylphenyl)carbamate (Compound No. 28)
(3-Benzyl-3-azabicyclo[3.2.1]oct-8-yl)methyl[2-(4-methylbenzyl)phenyl]carbamate (Compound No. 29),
N-(3-azabicyclo[3.2.1]oct-8-ylmethyl)biphenyl-2-carboxamide (Compound No. 30),
{3-[2-(1,3-Benzodioxol-5-yl)ethyl]-3-azabicyclo[3.2.1]oct-8-yl}methyl (2-benzylphenyl)carbamate (Compound No. 31),
(3-Benzyl-3-azabicyclo[3.2.1]oct-8-yl)methyl(4-chlorobenzyl)phenylcarbamate (Compound No. 32),
3-Azabicyclo[3.2.1]oct-8-ylmethyl (4-fluorobenzyl)phenylcarbamate (Compound No. 33),
(3-Benzyl-3-azabicyclo[3.2.1]oct-8-yl)methyl benzyl(4-chlorophenyl)carbamate (Compound No. 34),
Hydrochloride salt of 3-azabicyclo[3.2.1]oct-8-ylmethyl (2-fluorobenzyl)(3-fluorophenyl)carbamate (Compound No. 35),
(3-Benzyl-3-azabicyclo[3.2.1]oct-8-yl)methyl benzyl(3-fluorophenyl)carbamate (Compound No. 36),
(3-Benzyl-3-azabicyclo[3.2.1]oct-8-yl)methyl(cyclopentylmethyl)phenylcarbamate (Compound No. 37),
(3-Benzyl-3-azabicyclo[3.2.1]oct-8-yl)methyl(3,5-difluorobenzyl)(3-fluorophenyl)carbamate (Compound No. 38),
(3-Benzyl-3-azabicyclo[3.1.0]hex-1-yl)methyl[2-(2-thienyl)phenyl]carbamate (Compound No. 39),
Tert-butyl 6-[({[(2-fluorobenzyl)(phenyl)amino]carbonyl}oxy)methyl]-3-azabicyclo[3.1.0]hexane-3-carboxylate (Compound No. 40),
(3-Benzyl-3-azabicyclo[3.1.0]hex-6-yl)methyl benzyl(phenyl)carbamate (Compound No. 41),
Tert-butyl 8-[({[(4-fluorobenzyl)(phenyl)amino]carbonyl}oxy)methyl]-3-azabicyclo[3.2.1]octane-3-carboxylate (Compound No. 42),
3-Azabicyclo[3.2.1]oct-8-ylmethyl (4-fluorobenzyl)phenylcarbamate (Compound No. 43),
Tert-butyl 8-[({[(2-fluorobenzyl)(3-fluorophenyl)amino]carbonyl}oxy)methyl]-3-azabicyclo[3.2.1]octane-3-carboxylate (Compound No. 44),
3-Azabicyclo[3.2.1]oct-8-ylmethyl (2-fluorobenzyl)(3-fluorophenyl)carbamate (Compound No. 45),
3-Azabicyclo[3.1.0]hex-6-ylmethyl biphenyl-2-ylcarbamate (Compound No. 46),
(3-Benzyl-3-azabicyclo[3.2.1]oct-8-yl)methyl(3-hydroxy-4-methoxyphenyl)phenylcarbamate (Compound No. 47),
(3-Benzyl-3-azabicyclo[3.2.1]oct-8-yl)methyl 1H-imidazol-4-yl(phenyl)carbamate (Compound No. 48),
(3-Benzyl-3-azabicyclo[3.2.1]oct-8-yl)methyl(4-tert-butylphenyl)(3-fluorophenyl)carbamate (Compound No. 49),
(3-Benzyl-3-azabicyclo[3.2.1]oct-8-yl)methyl(4-tert-butylphenyl)phenylcarbamate (Compound No. 50),
(3-Benzyl-3-azabicyclo[3.2.1]oct-8-yl)methyl(3,5-difluorophenyl)phenylcarbamate (Compound No. 51),
(3-Benzyl-3-azabicyclo[3.2.1]oct-8-yl)methyl(3,4-difluorophenyl)(3-fluorophenyl)carbamate (Compound No. 52),
(3-Benzyl-3-azabicyclo[3.2.1]oct-8-yl)methyl(3,4-difluorophenyl)phenylcarbamate (Compound No. 53),
(3-Benzyl-3-azabicyclo[3.2.1]oct-8-yl)methyl(3-fluorophenyl)[4-(trifluoromethyl)phenyl]carbamate (Compound No. 54),
(3-Benzyl-3-azabicyclo[3.2.1]oct-8-yl)methyl phenyl[4-(trifluoromethyl)phenyl]carbamate (Compound No. 55),
(3-Benzyl-3-azabicyclo[3.2.1]oct-8-yl)methyl(3-fluorophenyl)(4-hydroxyphenyl)carbamate (Compound No. 56),
(3-Benzyl-3-azabicyclo[3.2.1]oct-8-yl)methyl(3-fluorophenyl)(3-hydroxy-4-methoxyphenyl)carbamate (Compound No. 57),
(3-Benzyl-3-azabicyclo[3.2.1]oct-8-yl)methyl(2-ethoxyphenyl)carbamate (Compound No. 58),
(3-Benzyl-3-azabicyclo[3.2.1]oct-8-yl)methyl(2-hydroxy-3-methoxyphenyl)carbamate (Compound No. 59),
(3-Benzyl-3-azabicyclo[3.2.1]oct-8-yl)methyl(3,4-dimethoxyphenyl)carbamate (Compound NO. 60),
(3-Benzyl-3-azabicyclo[3.2.1]oct-8-yl)methyl biphenyl-2-ylcarbamate (Compound No. 61),
(3-Benzyl-3-azabicyclo[3.2.1]oct-8-yl)methyl(4-phenoxyphenyl)carbamate (Compound No. 62),
(3-Benzyl-3-azabicyclo[3.2.1]oct-8-yl)methyl biphenyl-4-ylcarbamate (Compound No. 63),
(3-Benzyl-3-azabicyclo[3.2.1]oct-8-yl)methyl[2-(4-methoxybenzyl)phenyl]carbamate (Compound No. 64),
(3-Benzyl-3-azabicyclo[3.2.1]oct-8-yl)methyl[2-(3-methoxybenzoyl)phenyl]carbamate (Compound No. 65),
Hydrochloride salt of 3-azabicyclo[3.2.1]oct-8-yl)methyl(2-benzoylphenyl)carbamate (Compound No. 66),
(3-Benzyl-3-azabicyclo[3.2.1]oct-8-yl)methyl[2-(4-methylbenzoyl)phenyl]carbamate (Compound No. 67),
3-Benzyl-3-azabicyclo[3.2.1]oct-8-yl benzyl(2-fluorophenyl)carbamate (Compound No. 68),
Hydrochloride salt of 3-azabicyclo[3.2.1]oct-8-ylmethyl phenyl[3-(trifluoromethyl)benzyl]carbamate (Compound No. 69),
3-benzyl-3-azabicyclo[3.2.1]oct-8-yl (2-fluorobenzyl)(3-fluorophenyl)carbamate (Compound No. 70),
3-Benzyl-3-azabicyclo[3.2.1]oct-8-yl (4-methylbenzyl)phenylcarbamate (Compound No. 71),
3-Benzyl-3-azabicyclo[3.2.1]oct-8-yl (4-fluorobenzyl)phenylcarbamate (Compound No. 72),
3-Benzyl-3-azabicyclo[3.2.1]oct-8-yl benzyl(4-fluorophenyl)carbamate (Compound No. 73),
3-Benzyl-3-azabicyclo[3.2.1]oct-8-yl benzyl(4-chlorophenyl)carbamate (Compound No. 74),
3-Benzyl-3-azabicyclo[3.2.1]oct-8-yl (4-chlorobenzyl)phenylcarbamate (Compound No. 75),
N-[(3-benzyl-3-azabicyclo[3.2.1]oct-8-yl)methyl]-2-phenoxybenzamide (Compound No. 76),
N-[(3-Benzyl-3-azabicyclo[3.2.1]oct-8-yl)methyl]-4-phenoxybenzamide (Compound No. 77),
N-[(3-Benzyl-3-azabicyclo[3.2.1]oct-8-yl)methyl]-2-biphenyl-4-yl-N-methylacetamide (Compound No. 78),
N-[(3-Benzyl-3-azabicyclo[3.2.1]oct-8-yl)methyl]-4-cyclohexyl-N-methylbenzamide (Compound No. 79),
N-[(3-benzyl-3-azabicyclo[3.2.1]oct-8-yl)methyl]-4-cyclohexylbenzamide (Compound No. 80),
N-[(3-benzyl-3-azabicyclo[3.2.1]oct-8-yl)methyl]-N-methylbiphenyl-4-carboxamide (Compound No. 81),
N-[(3-Benzyl-3-azabicyclo[3.2.1]oct-8-yl)methyl]-4′-(trifluoromethyl)biphenyl-2-carboxamide (Compound No. 82),
N-[(3-Benzyl-3-azabicyclo[3.2.1]oct-8-yl)methyl]-N-methylbiphenyl-2-carboxamide (Compound No. 83),
N-[(3-Benzyl-3-azabicyclo[3.2.1]oct-8-yl)methyl]-2-biphenyl-4-ylacetamide (Compound No. 84),
N-[(3-Benzyl-3-azabicyclo[3.2.1]oct-8-yl)methyl]-4-cyclohexyl-N-methylbenzamide (Compound No. 85),
N-[(3-Benzyl-3-azabicyclo[3.2.1]oct-8-yl)methyl]biphenyl-4-carboxamide (Compound No. 86),
N-(3-Azabicyclo[3.2.1]oct-8-ylmethyl)-N-methyl-4′-(trifluoromethyl)biphenyl-2-carboxamide (Compound No. 87),
N-(3-Azabicyclo[3.2.1]oct-8-ylmethyl)-4-cyclohexylbenzamide (Compound No. 88),
N-(3-Azabicyclo[3.2.1]oct-8-ylmethyl)-4′-(trifluoromethyl)biphenyl-4-carboxamide (Compound No. 89),
N-(3-Azabicyclo[3.2.1]oct-8-ylmethyl)biphenyl-4-carboxamide (Compound No. 90),
N-(3-Azabicyclo[3.2.1]oct-8-ylmethyl)-2-biphenyl-4-yl-N-methylacetamide (Compound No. 91),
N-(3-Azabicyclo[3.2.1]oct-8-ylmethyl)-2-phenoxybenzamide (Compound No. 92) or
N-(3-Azabicyclo[3.2.1]oct-8-ylmethyl)-3-benzyl-N-methylbenzamide (Compound No. 93).

In other aspects, provided are pharmaceutical compositions comprising a therapeutically effective amount of a compound described herein and one or more pharmaceutically acceptable carriers, excipients or diluents.

Pharmaceutical compositions can include one or more of the following embodiments. For example, pharmaceutical compositions can further comprise one or more therapeutic ingredients selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or mixtures thereof.

In another aspect, provided are methods of treating or preventing a disease or disorder of the respiratory, urinary or gastrointestinal system, wherein the disease or disorder is mediated through muscarinic receptors in mammal comprising administering to a patient in need thereof a therapeutically effective amount of a compound described herein. The disease or disorder of the respiratory, urinary or gastrointestinal system can be urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

In yet another aspect, provided are methods of preparing a compound of Formula VI or a compound of Formula V comprising the steps of:

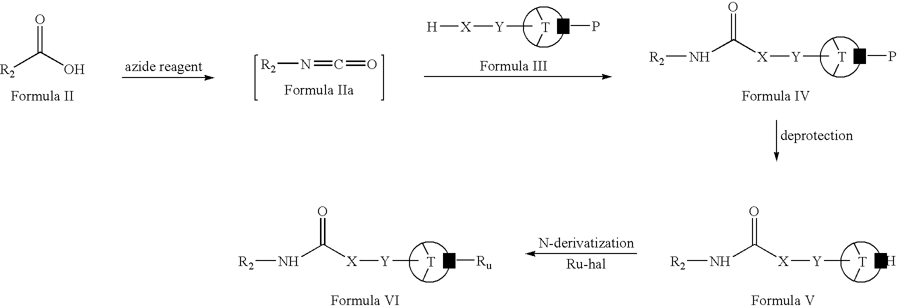

a) reacting a compound of Formula II with an azide reagent to form a compound of Formula IIa,
b) reacting the compound of Formula IIa with a compound of Formula III to form a compound of Formula IV,
c) deprotecting the compound of Formula IV to form a compound of Formula V, and
d) optionally N-derivatizing a compound of Formula V with a compound of Formula Rᵤ-hal to form a compound of Formula VI,

wherein

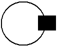

T, Q, n, X, Rₛ, Y, Rᵤ, R₃, Rₓ, R_{y}, and R₂ are the same as defined herein.

In yet another aspect, provided are methods of preparing a compound of Formula XI or a compound of Formula XIa comprising the steps of:

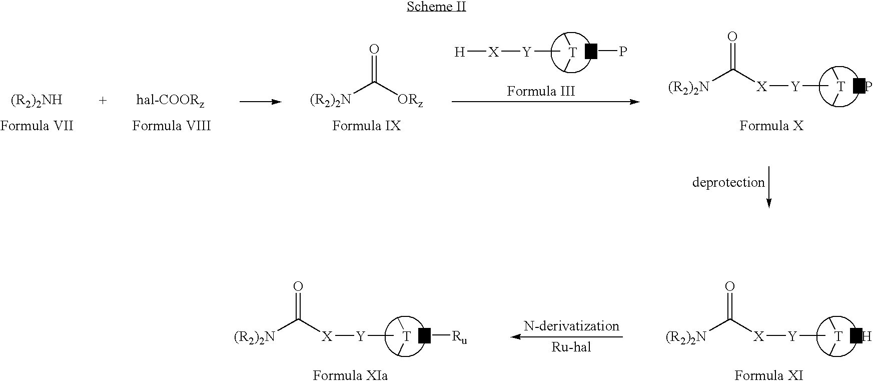

a) condensing a compound of Formula VII with compound of Formula VIII to form a compound of Formula IX,
b) reacting a compound of Formula IX with compound of Formula III to form a compound of Formula X,
c) deprotecting a compound of Formula X to form a compound of Formula XI, and
d) optionally N-derivatizing a compound of Formula XI with a compound of Formula Rᵤ-hal to form a compound of Formula XIa,

wherein

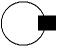

T, Q, n, X, Rₛ, Y, Rᵤ, R₃, Rₓ, R_{y}, R₂, P, R_{z} and hal are the same as defined herein.

In yet another aspect, provided are methods of preparing a compound of Formula XIII or a compound of Formula XIIIa comprising the steps of:

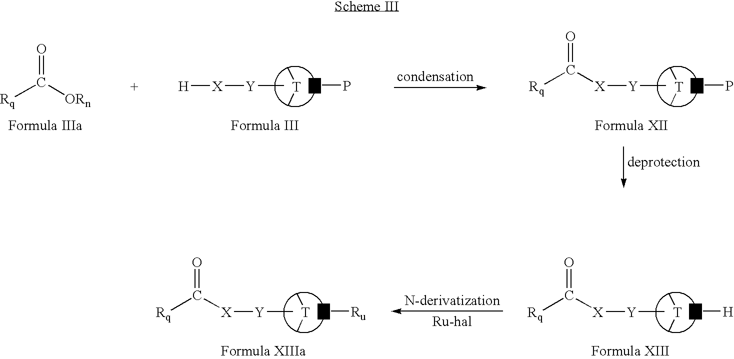

a) condensing a compound of Formula IIIa with a compound of Formula III to form a compound of Formula XII;
b) deprotecting a compound of Formula XII to form a compound of Formula XIII, and
c) optionally N-derivatizing a compound of Formula XIII with a compound of Formula Rᵤ-hal to form a compound of Formula XIIIa,

wherein

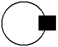

T, Q, n, X, Rₛ, Y, Rᵤ, R₃, Rₓ, R_{y}, R₂R_{q}, Rₙ, P and hal are the same as defined herein.

In another aspect, provided are methods of preparing a compound of Formula XVII or a compound of Formula XVIII comprising the steps of:

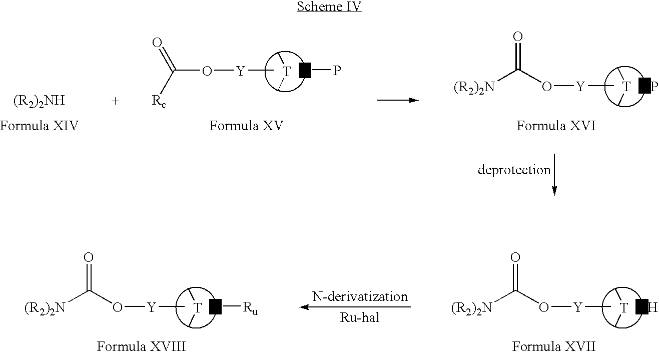

a) condensing a compound of Formula XIV with a compound of Formula XV to form a compound of Formula XVI;
b) deprotecting a compound of Formula XVI to form a compound of Formula XVII; and
c) N-derivatizing a compound of Formula XVII with a compound of Formula Rᵤ-hal to form a compound of Formula XVIII,

wherein

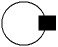

T, Q, n, X, Rₛ, Y, Rᵤ, R₃, Rₓ, R_{y}, R₂R_{q}, Rₙ, P and R_{c} are the same as defined herein.

Other aspects will be set forth in the description which follows, and in part will be apparent from the description or may be learnt by the practice of the invention.

### DETAIL DESCRIPTION OF THE INVENTION

In one aspect, provided are compounds having the structure of Formula I

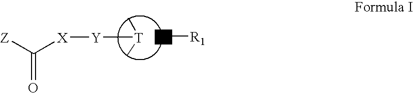

and pharmaceutically accepted salts, pharmaceutically acceptable solvates, enantiomers, diastereomers, polymorphs or N-oxides thereof, wherein

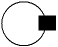

represents a nitrogen containing cyclic ring have 4-8 carbons;
T is a bridging group selected from —(CH₂)ₙ—, —CH(Q)CH₂—, —CH₂CH(Q)CH₂—, —CH(Q)—, —CH₂—O—CH₂— or —CH₂—NH—CH₂—,

wherein

the bridging group is attached to two carbon atoms of the ring

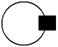

Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,

wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,

wherein when Y is no atom then X is directly attached to the ring

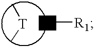

Z is —NHR₂, —N(R₂)₂, aryl or cycloalkyl,

wherein
R₂ is independently selected from alkyl, aryl, aralkyl, heteroaryl, cycloalkyl, heterocyclyl, heterocyclylalkyl or heteroarylalkyl; and
R₁ is selected from hydrogen, aralkyl or Rᵤ,

wherein
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
   wherein
   R₃ is alkyl, aryl, heteroaryl, heterocyclyl, cycloalkyl, aralkyl, heteroarylalkyl, heterocyclylalkyl or —NRₓR_{y}, and
   Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring.

In another aspect, provided are methods for the treatment or prophylaxis of a disease or disorder of the respiratory, urinary or gastrointestinal system, wherein the disease or disorder is mediated through muscarinic receptors, comprising administering one or more compounds described herein to an animal or a human in need thereof.

Diseases or disorders of the respiratory system include, for example, bronchial asthma, chronic obstructive pulmonary disorders (COPD), pulmonary fibrosis, and the like. Diseases or disorders of the urinary system include, for example, urinary incontinence, lower urinary tract symptoms (LUTS), and the like. Diseases or disorders of the gastrointestinal system include, for example, irritable bowel syndrome, obesity, diabetes or gastrointestinal hyperkinesis.

In yet another aspect, provided are processes for preparing the compounds described herein.

The compounds described herein can exhibit significant potency in terms of their activity, as determined by in vitro receptor binding and functional assays and in vivo experiments using anaesthetized rabbits. Compounds that were found active in vitro were tested in vivo. Pharmaceutical compositions for treating diseases or disorders associated with muscarinic receptors are provided. In addition, compounds can be administered by any route including, for example, orally or parenterally.

The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein R_{α} can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π} {wherein R_{λ} and R_{π}, are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ} are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(═O)NR_{λ}R_{π}, —O—C(═O)NR_{λ})R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ})R_{π} and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “alkylene,” as used herein, refers to a diradical branched or unbranched saturated hydrocarbon chain having from 1 to 6 carbon atoms and one or more hydrogen can optionally be substituted with alkyl, hydroxy, halogen or oximes. This term can be exemplified by groups such as methylene, ethylene, propylene isomers (e.g., —CH₂CH₂CH₂ and —CH(CH₃)CH₂) and the like. Alkylene may further be substituted with one or more substituents such as alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR_{ψ}, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, nitro, —S(O)ₘR_{λ} (wherein R_{λ}, R_{π}, m and R_{ψ} are the same as defined earlier). Unless otherwise constrained by the definition, all substituents may be further substituted by 1-3 substituents chosen from alkyl, alkenyl, alkynyl, carboxy, —COOR_{ψ}, —NR_{λ}R_{π}, —C(═O)NR_{λ}R_{π}, —OC(═O)NR_{λ}R_{π}, —NHC(═O)NR_{λ}R_{π}, hydroxy, alkoxy, halogen, CF₃, cyano, and —S(O)ₘR_{ψ} (wherein R_{λ}, R_{π}, m and R_{ψ} are the same as defined earlier). Alkylene can also be optionally interrupted by 1-5 atoms of groups independently chosen from oxygen, sulfur and —NR_{α} (wherein R_{α} is the same as defined earlier). Unless otherwise constrained by the definition, all substituents may be further substituted by 1-3 substituents selected from hydrogen, alkyl, cycloalkyl, alkenyl, alkynyl, aryl, acyl, aralkyl, alkoxy, hydroxy, carboxy, —C(═O)OR_{ψ}, halogen, CF₃, cyano, —NR_{λ}R_{π}, —S(O)ₘR_{ψ}, —C(═O)NR_{λ}R_{π}, —OC(═O)NR_{λ}R_{π}, —CONH—, —C═O or —C═NOH (wherein R_{λ}, R_{π}, m and R_{ψ} are the same as defined earlier).

The term “alkoxy” denotes the group O-alkyl, wherein alkyl is the same as defined above.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo[2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —NHC(═O)R_{λ}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “carboxy” as defined herein refers to —C(═O)OH.

The term “aryloxy” denotes the group O-aryl, wherein aryl is as defined above.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}, —NHC(═O)NR_{λ}R_{π}, —SOₘR_{ψ}, —O—C(═O)NR_{λ}R_{π}, —O—C(═O)R_{λ}, or —O—C(═O)OR_{λ} (wherein m, R_{ψ}, R_{λ} and R_{π}, are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π}, are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

The term “heteroarylalkyl,” unless otherwise specified, refers to heteroaryl (wherein heteroaryl is same as defined earlier) linked through alkyl (wherein alkyl is the same as defined above) portion and the alkyl portion contains carbon atoms from 1-6.

The term “heterocyclylalkyl,” unless otherwise specified, refers to heterocyclyl (wherein heterocyclyl is same as defined earlier) linked through alkyl (wherein alkyl is the same as defined above) portion and the alkyl portion contains carbon atoms from 1-6.

The term “acyl,” unless otherwise specified refers to —C(═O)R″ wherein R″ is selected from the group hydrogen, alkyl, cycloalkyl, aryl, aralkyl, heteroaryl, heterocyclyl, heteroarylalkyl or heterocyclylalkyl.

The term “thiocarbonyl,” unless otherwise specified, refers to —C(═S)H.

The term “substituted thiocarbonyl,” unless otherwise specified, refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

The term “leaving group,” unless otherwise specified, generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “protecting groups,” unless otherwise specified, is used herein to refer to known moieties, which have the desirable property of preventing specific chemical reaction at a site on the molecule undergoing chemical modification intended to be left unaffected by the particular chemical modification. Also the term protecting group, unless or other specified may be used with groups such as hydroxy, amino, carboxy and example of such groups are found in T. W. Greene and P. G. M. Wuts, “Protective Groups in Organic Synthesis”, 2^{nd} Edn. John Wiley and Sons, New York, N.Y., which is incorporated herein by reference. The species of the carboxylic protecting groups, amino protecting groups or hydroxy protecting group employed is not so critical so long as the derivatised moiety/moieties is/are stable to conditions of subsequent reactions and can be removed at the appropriate point without disrupting the remainder of the molecule.

The term “pharmaceutically acceptable salts,” unless otherwise specified, refers to derivatives of compounds that can be modified by forming their corresponding acid or base salts. Examples of pharmaceutically acceptable salts include, but are not limited to, mineral or organic acids salts of basic residues (such as amines) or alkali or organic salts of acidic residues (such as carboxylic acids), and the like. Pharmaceutically acceptable salts may also be formed by complete derivatization of the amine moiety e,g. quaternary ammonium salts. The quaternary ammonium salts of the compound of Formula I can be prepared by reaction of compound of Formula I with Q-Z wherein (Q is selected from alkyl, alkenyl, alkynyl, cycloalkyl, heterocyclyl, aryl, heteroaryl, aralkyl, heteroarylalkyl or heterocyclylalkyl and Z is an anion disclosed in International Journal of pharmaceutics, 33 (1986), page 202, for example, but not limited to, tartarate, chloride, bromide, iodide, sulphate, phosphate, nitrate, carbonate, fumarate, glutamate, citrate, methanesulphonate, benzenesulphonate, maleate or succinate).

The compounds disclosed herein may be prepared by methods represented by the reaction sequences, for example, as generally shown in Schemes I, II and III

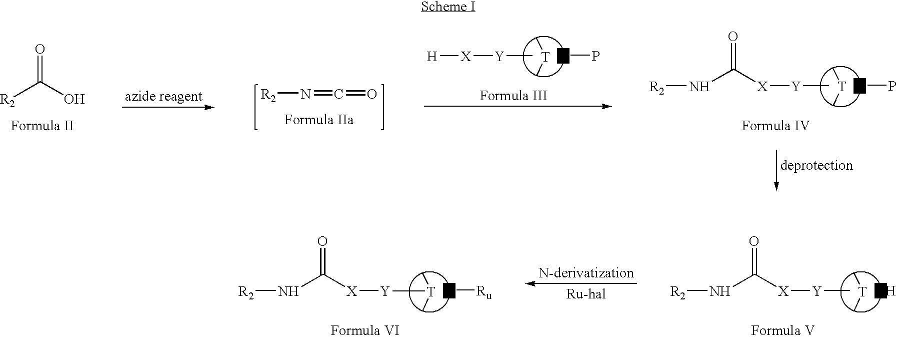

Compounds of Formula V and VI may be prepared, for example, by the reaction sequence as shown in Scheme I. In particular, compounds of Formula II (wherein R₂ is the same as defined earlier) can be reacted with one or more azide reagents to form compounds of Formula IIa. This reaction can be carried out in-situ. Compounds of Formula IIa can be reacted with compounds of Formula III (wherein P is a protecting group, for example, aralkyl, —C(═O)Oaralkyl, —C(═O)OC(CH₃)₃, —C(═O)OC(CH₃)₂CHBr₂ or C(═O)OC(CH₃)₂CCl₃; and T, X and Y are the same as defined earlier) to form compounds of Formula IV. Compounds of Formula VI can be deprotected to form compounds of Formula V. Compounds of Formula V can be N-derivatized with compounds of Formula Rᵤ-hal (wherein Rᵤ is same as defined earlier and hal is Br, Cl or I) to form compounds of Formula VI.

Compounds of Formula II can be reacted with one or more azide reagents including, for example, diphenyl phosphonic azide, sodium azide or mixtures thereof. This reaction can be carried out in-situ.

Compounds of Formula IIa can be reacted with compound of Formula III in one or more bases. Suitable bases include, for example, triethylamine, pyridine, diisopropylethylamine, N-methylmorpholine or mixtures thereof. This reaction can also be carried out in one or more organic solvents (for example, toluene, heptane, xylene or mixtures thereof).

Compounds of Formula IV (wherein P is —C(═O)OC(CH₃)₃ or —C(═O)OC(CH₃)₂CHBr₂) can be deprotected in an acid-alcohol solution (for example, as solution of hydrochloric acid in methanol, ethanol, propanol, isopropylalcohol, ethyl acetate, ether or mixtures thereof) or trifluoroacetic acid in dichloromethane. Alternatively, the deprotection reaction can be carried out in the presence of one or more supernucleophiles (for example, lithium cobalt (I) phthalocyanine, zinc and acetic acid, cobalt phthalocyanine or mixtures thereof).

Compounds of Formula IV (wherein P is —C(═O)OC(CH₃)₂CCl₃) can be deprotected in the presence of one or more supernucleophiles (for example, lithium cobalt (I) phthalocyanine, zinc and acetic acid, cobalt phthalocyanine or mixtures thereof).

Compounds of Formula IV (wherein P is aralkyl or —C(═O)Oaralkyl) can be deprotected in the presence of one or more deprotecting agents (for example, by hydrogenation). Suitable deprotecting agents include, for example, palladium on carbon in presence of hydrogen gas or palladium on carbon with a source of hydrogen gas (for example, ammonium formate, cyclohexene or formic acid). The deprotection can also be carried out in one or more organic solvents (for example, ethyl acetate, methanol, ethanol, propanol, isopropylalcohol or mixtures thereof).

Alternatively, compounds of Formula IV (wherein P is —C(═O)Oaralkyl) can be deprotected in an alkaline solution. Suitable alkaline solutions comprise one or more bases (for example, potassium hydroxide, sodium hydroxide, lithium hydroxide or mixtures thereof) and one or more solvents (for example, methanol, ethanol propanol, diethylether, isopropylalcohol or mixtures thereof).

Compounds of Formula V can be N-derivatized with compounds of Formula Ru-hal in the presence of one or more bases (for example, potassium carbonate, sodium carbonate, sodium bicarbonate or mixtures thereof). The N-derivatization can also be carried out in one or more organic solvents (for example, acetonitrile, dichloromethane, chloroform, carbon tetrachloride or mixtures thereof).

Alternatively, compounds of Formula V can be N-derivatized by reductive amination. The reductive amination can be carried out in the presence of one or more reducing agents (for example, sodium cyanoborohydride, sodium triacetoxyborohydride or mixtures thereof). The reductive amination can also be carried out in one or more organic solvents (for example, acetonitrile, dichloromethane, tetrahydrofuran or mixtures thereof).

Exemplary compounds include, for example:
(3-Benzyl-3-azabicyclo[3.1.0]hex-6-yl)methyl biphenyl-2-ylcarbamate (Compound No. 1),
N-[(3-benzyl-3-azabicyclo[3.1.0]hex-6-yl)methyl]-N′-biphenyl-2-ylurea (Compound No. 2),
Tartarate salt of 3-azabicyclo[3.1.0]hex-6-ylmethyl biphenyl-2-ylcarbamate (Compound No. 3),
3-Benzyl-3-azabicyclo[3.2.1]oct-8-yl biphenyl-2-ylcarbamate (Compound No. 4),
3-azabicyclo[3.2.1]oct-8-yl biphenyl-2-ylcarbamate (Compound No. 5),
2-Benzyl-2-azabicyclo[2.2.1]hept-7-yl biphenyl-2-ylcarbamate (Compound No. 6),
(3-Benzyl-3-azabicyclo[3.2.1]oct-8-yl)methyl biphenyl-2-ylcarbamate (Compound No. 7),
(3-Benzyl-3-azabicyclo[3.1.0]hex-1-yl)methyl biphenyl-2-ylcarbamate (Compound No. 8),
(3-Benzyl-3-azabicyclo[3.1.0]hex-6-yl)methyl[2-(2-thienyl)phenyl]carbamate (Compound No. 9),
3-azabicyclo[3.1.0]hex-6-ylmethyl[2-(2-thienyl)phenyl]carbamate (Compound No. 10),
(3-Benzyl-3-azabicyclo[3.1.0]hex-6-yl)methyl(2′,4′-difluorobiphenyl-2-yl)carbamate (Compound No. 11),
(3-Benzyl-3-azabicyclo[3.1.0]hex-1-yl)methyl(2′,4′-difluorobiphenyl-2-yl)carbamate (Compound No. 12),
(3-Benzyl-3-azabicyclo[3.1.0]hex-6-yl)methyl(2′,4′-dimethoxybiphenyl-2-yl)carbamate (Compound No. 13),
3-Azabicyclo[3.1.0]hex-6-ylmethyl (2′,4′-difluorobiphenyl-2-yl)carbamate (Compound No. 17),
3-Azabicyclo[3.1.0]hex-6-ylmethyl (2′,4′-dimethoxybiphenyl-2-yl)carbamate (Compound No. 18),
3-Benzyl-3-azabicyclo[3.1.0]hex-6-yl)methyl[2-(1,3-benzodioxol-5-yl)phenyl]carbamate (Compound No. 19),
(3-Benzyl-3-azabicyclo[3.2.1]oct-8-yl)methyl(2-benzylphenyl)carbamate (Compound No. 20),
3-Azabicyclo[3.1.0]hex-6-ylmethyl (2-benzylphenyl)carbamate (Compound No. 22),
(3-benzyl-3-azabicyclo[3.2.1]oct-8-yl)methyl(2-benzoylphenyl)carbamate (Compound No. 28)
(3-Benzyl-3-azabicyclo[3.2.1]oct-8-yl)methyl[2-(4-methylbenzyl)phenyl]carbamate (Compound No. 29),
{3-[2-(1,3-Benzodioxol-5-yl)ethyl]-3-azabicyclo[3.2.1]oct-8-yl}methyl (2-benzylphenyl)carbamate (Compound No. 31),
(3-Benzyl-3-azabicyclo[3.1.0]hex-1-yl)methyl[2-(2-thienyl)phenyl]carbamate (Compound No. 39),
3-Azabicyclo[3.1.0]hex-6-ylmethyl biphenyl-2-ylcarbamate (Compound No. 46),
(3-Benzyl-3-azabicyclo[3.2.1]oct-8-yl)methyl(2-ethoxyphenyl)carbamate (Compound No. 58),
(3-Benzyl-3-azabicyclo[3.2.1]oct-8-yl)methyl(2-hydroxy-3-methoxyphenyl)carbamate (Compound No. 59),
(3-Benzyl-3-azabicyclo[3.2.1]oct-8-yl)methyl(3,4-dimethoxyphenyl)carbamate (Compound No. 60),
(3-Benzyl-3-azabicyclo[3.2.1]oct-8-yl)methyl biphenyl-2-ylcarbamate (Compound No. 61),
(3-Benzyl-3-azabicyclo[3.2.1]oct-8-yl)methyl(4-phenoxyphenyl)carbamate (Compound No. 62),
(3-Benzyl-3-azabicyclo[3.2.1]oct-8-yl)methyl biphenyl-4-ylcarbamate (Compound No. 63),
(3-Benzyl-3-azabicyclo[3.2.1]oct-8-yl)methyl[2-(4-methoxybenzyl)phenyl]carbamate (Compound No. 64),
(3-Benzyl-3-azabicyclo[3.2.1]oct-8-yl)methyl[2-(3-methoxybenzoyl)phenyl]carbamate (Compound No. 65),
Hydrochloride salt of (3-azabicyclo[3.2.1]oct-8-yl)methyl(2-benzoylphenyl)carbamate (Compound No. 66),
(3-Benzyl-3-azabicyclo[3.2.1]oct-8-yl)methyl[2-(4-methylbenzoyl)phenyl]carbamate (Compound No. 67)
   and pharmaceutically accepted salts, pharmaceutically acceptable solvates, enantiomers, diastereomers, polymorphs or N-oxides thereof.

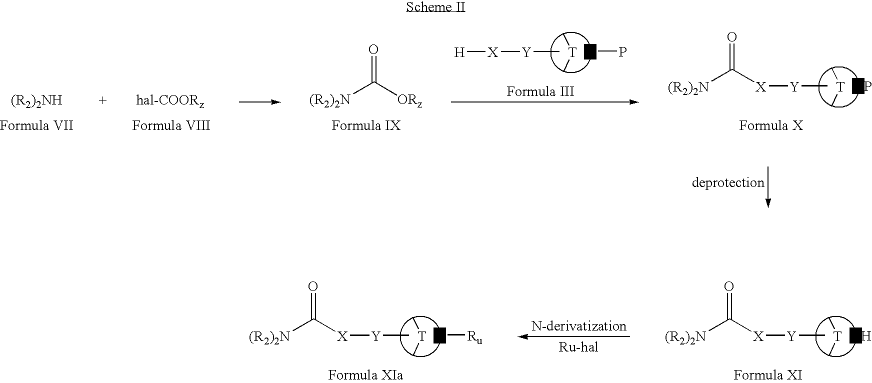

Compounds of Formulae X, XI and XIa may be prepared, for example, by the following reaction sequence as given in Scheme II. Compounds of Formula VII (wherein R₂ is the same as defined earlier) can be condensed with compounds of Formula VIII to give compound of Formula IX (wherein R_{z} is alkyl or aryl). Compounds of Formula IX can be reacted with compounds of Formula III (wherein X, Y, T and P are the same as defined earlier) to form compounds of Formula X. Compounds of Formula X can be deprotected to form compounds of Formula XI. Compounds of Formula XI can be N-derivatized with compounds of Formula Rᵤ-hal (wherein Rᵤ and hal are the same as defined earlier) to form compounds of Formula XIa.

Compounds of Formula VII can be reacted with compounds of Formula VIII in the presence of one or more bases (for example, triethylamine, pyridine, diisopropylethylamine or mixtures thereof). The reaction can also be carried out in one or more organic solvents (for example, tetrahydrofuran, dioxane, dimethylformamide, diethylether, dichloromethane or mixtures thereof).

Compounds of Formula IX can be reacted (by condensation) with compounds of Formula III. The reaction can be carried out in presence of one or more bases (for example, sodium hydride, lithium diisopropylamide, pyridine or mixtures thereof). The reaction can also be carried out in one or more organic solvents (for example, toluene, heptane, xylene or mixtures thereof).

Compounds of Formula X can be deprotected to form compounds of Formula XI by following the procedure described in Scheme I for the deprotection of compound of Formula IV to compound of Formula V. In particular, Compounds of Formula X (wherein P is —C(═O)OC(CH₃)₃ or —C(═O)OC(CH₃)₂CHBr₂) can be deprotected in an acid-alcohol solution (for example, as solution of hydrochloric acid in methanol, ethanol, propanol, isopropylalcohol, ethyl acetate, ether or mixtures thereof) or trifluoroacetic acid in dichloromethane. Alternatively, the deprotection reaction can be carried out in the presence of one or more supernucleophiles (for example, lithium cobalt (I) phthalocyanine, zinc and acetic acid, cobalt phthalocyanine or mixtures thereof).

Compounds of Formula X (wherein P is —C(═O)OC(CH₃)₂CCl₃) can be deprotected in the presence of one or more supernucleophiles (for example, lithium cobalt (I) phthalocyanine, zinc and acetic acid, cobalt phthalocyanine or mixtures thereof).

Compounds of Formula X (wherein P is aralkyl or —C(═O)Oaralkyl) can be deprotected in the presence of one or more deprotecting agents (for example, by hydrogenation). Suitable deprotecting agents include, for example, palladium on carbon in presence of hydrogen gas or palladium on carbon with a source of hydrogen gas (for example, ammonium formate, cyclohexene or formic acid). The deprotection can also be carried out in one or more organic solvents (for example, ethyl acetate, methanol, ethanol, propanol, isopropylalcohol or mixtures thereof).

Alternatively, compounds of Formula X (wherein P is —C(═O)Oaralkyl) can be deprotected in an alkaline solution. Suitable alkaline solutions comprise one or more bases (for example, potassium hydroxide, sodium hydroxide, lithium hydroxide or mixtures thereof) and one or more solvents (for example, methanol, ethanol propanol, diethylether, isopropylalcohol or mixtures thereof).

Compounds of Formula XI can be N-derivatized with compounds of Formula Ru-hal in the presence of one or more bases (for example, potassium carbonate, sodium carbonate, sodium bicarbonate or mixtures thereof). The reaction can also be carried out in one or more organic solvents (for example, acetonitrile, dichloromethane, chloroform, carbon tetrachloride or mixtures thereof).

Alternatively, compounds of Formula XI can be N-derivatized in the presence of one or more reducing agents (for example, sodium cyanoborohydride, sodium triacetoxyborohydride or mixtures thereof). The N-derivatization can also be carried out by reductive amination. The reaction can be carried out in one or more organic solvents (for example, acetonitrile or dichloromethane, tetrahydrofuran or mixtures thereof).

Exemplary compounds include, for example:
3-Azabicyclo[3.1.0]hex-6-ylmethyl (2-fluorobenzyl)phenylcarbamate (Compound No. 14),
(3-Benzyl-3-azabicyclo[3.1.0]hex-6-yl)methyl benzyl(3-fluorophenyl)carbamate (Compound No. 16),
(3-Benzyl-3-azabicyclo[3.2.1]oct-8-yl)methyl benzyl (4-fluorophenyl)carbamate. (Compound No. 24),
(3-Benzyl-3-azabicyclo[3.2.1]oct-8-yl)methyl(2-fluorobenzyl)phenyl carbamate. (Compound No. 25),
(3-Benzyl-3-azabicyclo[3.2.1]oct-8-yl)methyl benzyl(phenyl)carbamate (Compound No. 26),
(3-Benzyl-3-azabicyclo[3.2.1]oct-8-yl)methyl(4-methylbenzyl)phenyl carbamate (Compound No. 27),
(3-Benzyl-3-azabicyclo[3.2.1]oct-8-yl)methyl(4-chlorobenzyl)phenylcarbamate (Compound No. 32),
3-Azabicyclo[3.2.1]oct-8-ylmethyl (4-fluorobenzyl)phenylcarbamate (Compound No. 33),
(3-Benzyl-3-azabicyclo[3.2.1]oct-8-yl)methyl benzyl(4-chlorophenyl)carbamate (Compound No. 34),
Hydrochloride salt of 3-azabicyclo[3.2.1]oct-8-ylmethyl (2-fluorobenzyl)(3-fluorophenyl)carbamate (Compound No. 35),
(3-Benzyl-3-azabicyclo[3.2.1]oct-8-yl)methyl benzyl(3-fluorophenyl)carbamate (Compound No. 36),
(3-Benzyl-3-azabicyclo[3.2.1]oct-8-yl)methyl(cyclopentylmethyl)phenylcarbamate (Compound No. 37),
(3-Benzyl-3-azabicyclo[3.2.1]oct-8-yl)methyl(3,5-difluorobenzyl)(3-fluorophenyl)carbamate (Compound No. 38),
Tert-butyl 6-[({[(2-fluorobenzyl)(phenyl)amino]carbonyl}oxy)methyl]-3-azabicyclo[3.1.0]hexane-3-carboxylate (Compound No. 40),
(3-Benzyl-3-azabicyclo[3.1.0]hex-6-yl)methyl benzyl(phenyl)carbamate (Compound No. 41),
Tert-butyl 8-[({[(4-fluorobenzyl)(phenyl)amino]carbonyl}oxy)methyl]-3-azabicyclo[3.2.1]octane-3-carboxylate (Compound No. 42),
3-Azabicyclo[3.2.1]oct-8-ylmethyl (4-fluorobenzyl)phenylcarbamate (Compound No. 43),
Tert-butyl 8-[({[(2-fluorobenzyl)(3-fluorophenyl)amino]carbonyl}oxy)methyl]-3-azabicyclo[3.2.1]octane-3-carboxylate (Compound No. 44),
3-Azabicyclo[3.2.1]oct-8-ylmethyl (2-fluorobenzyl)(3-fluorophenyl)carbamate (Compound No. 45),
(3-Benzyl-3-azabicyclo[3.2.1]oct-8-yl)methyl(3-hydroxy-4-methoxyphenyl)phenylcarbamate (Compound No. 47),
(3-Benzyl-3-azabicyclo[3.2.1]oct-8-yl)methyl 1H-imidazol-4-yl(phenyl)carbamate (Compound No. 48),
(3-Benzyl-3-azabicyclo[3.2.1]oct-8-yl)methyl(4-tert-butylphenyl)(3-fluorophenyl)carbamate (Compound No. 49),
(3-Benzyl-3-azabicyclo[3.2.1]oct-8-yl)methyl(4-tert-butylphenyl)phenylcarbamate (Compound No. 50),
(3-Benzyl-3-azabicyclo[3.2.1]oct-8-yl)methyl(3,5-difluorophenyl)phenylcarbamate (Compound No. 51),
(3-Benzyl-3-azabicyclo[3.2.1]oct-8-yl)methyl(3,4-difluorophenyl)(3-fluorophenyl)carbamate (Compound No. 52),
(3-Benzyl-3-azabicyclo[3.2.1]oct-8-yl)methyl(3,4-difluorophenyl)phenylcarbamate (Compound No. 53),
(3-Benzyl-3-azabicyclo[3.2.1]oct-8-yl)methyl(3-fluorophenyl)[4-(trifluoromethyl)phenyl]carbamate (Compound No. 54),
(3-Benzyl-3-azabicyclo[3.2.1]oct-8-yl)methyl phenyl[4-(trifluoromethyl)phenyl]carbamate (Compound No. 55),
(3-Benzyl-3-azabicyclo[3.2.1]oct-8-yl)methyl(3-fluorophenyl)(4-hydroxyphenyl)carbamate (Compound No. 56),
(3-Benzyl-3-azabicyclo[3.2.1]oct-8-yl)methyl(3-fluorophenyl)(3-hydroxy-4-methoxyphenyl)carbamate (Compound No. 57),
3-Benzyl-3-azabicyclo[3.2.1]oct-8-yl benzyl(2-fluorophenyl)carbamate (Compound No. 68),
Hydrochloride salt of 3-azabicyclo[3.2.1]oct-8-ylmethyl phenyl[3-(trifluoromethyl)benzyl]carbamate (Compound No. 69),
3-benzyl-3-azabicyclo[3.2.1]oct-8-yl (2-fluorobenzyl)(3-fluorophenyl)carbamate (Compound No. 70),
3-Benzyl-3-azabicyclo[3.2.1]oct-8-yl (4-methylbenzyl)phenylcarbamate (Compound No. 71),
3-Benzyl-3-azabicyclo[3.2.1]oct-8-yl (4-fluorobenzyl)phenylcarbamate (Compound No. 72),
3-Benzyl-3-azabicyclo[3.2.1]oct-8-yl benzyl(4-fluorophenyl)carbamate (Compound No. 73),
3-Benzyl-3-azabicyclo[3.2.1]oct-8-yl benzyl(4-chlorophenyl)carbamate (Compound No. 74),
3-Benzyl-3-azabicyclo[3.2.1]oct-8-yl (4-chlorobenzyl)phenylcarbamate (Compound No. 75),
   and pharmaceutically accepted salts, pharmaceutically acceptable solvates, enantiomers, diastereomers, polymorphs or N-oxides thereof.

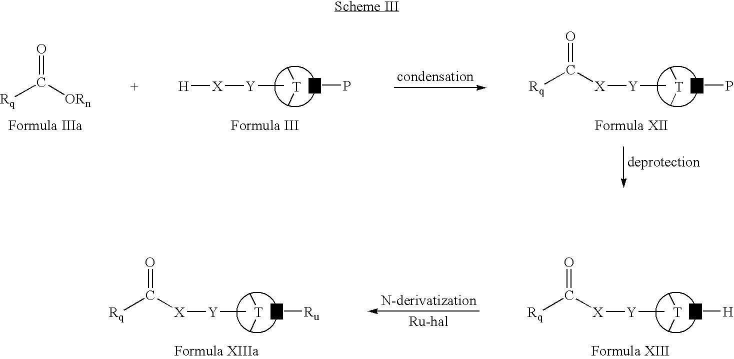

Compounds of Formulae XII, XIII and XIIIa may be prepared, for example, by the reaction sequence as shown in Scheme III. In particular, compounds of Formula IIIa (wherein R_{q} is aryl or cycloalkyl and Rₙ is hydrogen or alkyl) can be condensed with compounds of Formula III (wherein X, Y, T and P are the same as defined earlier) to form compounds of Formula XII. Compounds of Formula XII can be deprotected to form compounds of Formula XIII. Compounds of Formula XIII can be N-derivatized with compounds of Formula Rᵤ-hal (wherein Rᵤ and hal are the same as defined earlier) to form compounds of Formula XIIIa.

Compounds of Formula IIIa can be condensed with compounds of Formula III (wherein X is —O or —S; Y is the same as defined earlier and Rₙ is alkyl) in the presence of one or more bases (for example, sodium hydride, sodium methoxide or mixtures thereof) to form compounds of Formula XII. The reaction can also be carried out in one or more organic solvents (for example, toluene, benzene, hexane, heptane, xylene or mixtures thereof).

Compounds of Formula IIIa can be condensed with compounds of Formula III (wherein X is —O or —S; Y the same as defined earlier and Rₙ is hydrogen) in the presence of carbonyldiimidazole and one or more bases (for example, sodium hydride, triethylamine, N-ethyldiisopropylamine, pyridine or mixtures thereof). The reaction can also be carried out in one or more organic solvents (for example, dimethylformamide, tetrahydrofuran, diethylether, dioxane or mixtures thereof). Alternatively, compounds of Formula IIIa can be condensed with compounds of Formula III (wherein X is —O or —S; Y the same as defined earlier and Rₙ is hydrogen) in the presence of one or more bases (for example, 1,8-diazabicyclo[5.4.0]undecen-7-ene, 1,4-diazabicyclo[2.2.2]octane or mixtures thereof). The reaction can also be carried out in one or more organic solvents (for example, toluene, heptane, xylene or mixtures thereof).

Compounds of Formula IIIa can be condensed with compounds of Formula III (wherein X is —NRₛ; Y is alkylene; Rₙ is hydrogen; and Rₛ is the same as defined earlier) can be carried out in the presence of one or more base (for example, N-methylmorpholine, triethylamine, diisopropylethylamine, pyridine or mixtures thereof) and one or more condensing agents (for example, 1-(3-dimethylaminopropyl)-3-ethylcarbodiimide hydrochloride (EDC.HCl), dicyclohexylcarbodiimide or mixtures thereof). The reaction can also be carried out in one or more organic solvents (for example, dimethylformamide, tetrahydrofuran, diethyl ether, dioxane or mixtures thereof).

Compounds of Formula IIIa can be condensed with compounds of Formula III (wherein X is —NRs; Y is alkylene and Rₙ is alkyl) can be carried out in the presence of one or more reducing agents, for example, diisobutyl aluminum. The reaction can also be carried out in one or more organic solvents (for example, tetrahydrofuran, diethyl ether, dioxane, dimethylformamide or mixtures thereof).

Compounds of Formula XII (wherein P is aralkyl or —C(═O)Oaralkyl) can be deprotected can be carried out in the presence of one or more deprotecting agents. Suitable deprotecting agents include, for example, palladium on carbon in presence of hydrogen gas or palladium on carbon with a source of hydrogen gas (for example, ammonium formate solution, cyclohexene or formic acid). The reaction can also be carried out in the presence of one or more organic solvents (for example, methanol, ethanol, propanol, isopropylalcohol or mixtures thereof).

Alternatively, compounds of Formula XII (when P is —C(═O)Oaralkyl) can be deprotected in an alkaline solution. Suitable alkaline solutions comprise one or more bases (for example, potassium hydroxide, sodium hydroxide, lithium hydroxide or mixtures thereof) and one or more solvents (for example, methanol, ethanol propanol, diethylether, isopropylalcohol or mixtures thereof).

Compounds of Formula IV (wherein P is —C(═O)OC(CH₃)₃ or —C(═O)OC(CH₃)₂CHBr₂) can be deprotected in an acidic solution (for example, hydrochloric acid solution in one or more solvents, e.g., methanol, ethanol, propanol, isopropylalcohol, ethyl acetate, ether or mixtures thereof) or trifluoroacetic acid in dichloromethane. Alternatively, the deprotection reaction can be carried out in the presence of one or more supernucleophiles (for example, lithium cobalt (I) phthalocyanine, zinc and acetic acid, cobalt phthalocyanine or mixtures thereof).

Compounds of Formula XII (wherein P is —C(═O)OC(CH₃)₂CCl₃) can be deprotected in the presence of one or more supernucleophiles (for example, lithium cobalt (I) phthalocyanine, zinc and acetic acid, cobalt phthalocyanine or mixtures thereof).

Compounds of Formula XIII can be N-derivatized with compounds of Formula Ru-hal in the presence of one or more bases (for example, potassium carbonate, sodium carbonate, sodium bicarbonate or mixtures thereof). The reaction can also be carried out in one or more organic solvents (for example, acetonitrile, dichloromethane, chloroform, carbon tetrachloride or mixtures thereof).

Alternatively, compounds of Formula XIII can be N-derivatized by reductive amination in the presence of one or more reducing agents (for example, sodium cyanoborohydride, sodium triacetoxyborohydride or mixtures thereof). The reaction can also be carried out in one or more organic solvents (for example, acetonitrile or dichloromethane, tetrahydrofuran or mixtures thereof).

Exemplary compounds include, for example:
N-[(3-benzyl-3-azabicyclo[3.2.1]oct-8-yl)methyl]biphenyl-2-carboxamide (Compound No. 21),
2-Benzyl-N-[(3-benzyl-3-azabicyclo[3.2.1]oct-8-yl)methyl]benzamide (Compound No. 23),
N-(3-azabicyclo[3.2.1]oct-8-ylmethyl)biphenyl-2-carboxamide (Compound No. 30),
N-[(3-benzyl-3-azabicyclo[3.2.1]oct-8-yl)methyl]-2-phenoxybenzamide (Compound No. 76),
N-[(3-Benzyl-3-azabicyclo[3.2.1]oct-8-yl)methyl]-4-phenoxybenzamide (Compound No. 77),
N-[(3-Benzyl-3-azabicyclo[3.2.1]oct-8-yl)methyl]-2-biphenyl-4-yl-N-methylacetamide (Compound No. 78),
N-[(3-Benzyl-3-azabicyclo[3.2.1]oct-8-yl)methyl]-4-cyclohexyl-N-methylbenzamide (Compound No. 79),
N-[(3-benzyl-3-azabicyclo[3.2.1]oct-8-yl)methyl]-4-cyclohexylbenzamide (Compound No. 80),
N-[(3-benzyl-3-azabicyclo[3.2.1]oct-8-yl)methyl]-N-methylbiphenyl-4-carboxamide (Compound No. 81),
N-[(3-Benzyl-3-azabicyclo[3.2.1]oct-8-yl)methyl]-4′-(trifluoromethyl)biphenyl-2-carboxamide (Compound No. 82),
N-[(3-Benzyl-3-azabicyclo[3.2.1]oct-8-yl)methyl]-N-methylbiphenyl-2-carboxamide (Compound No. 83),
N-[(3-Benzyl-3-azabicyclo[3.2.1]oct-8-yl)methyl]-2-biphenyl-4-ylacetamide (Compound No. 84),
N-[(3-Benzyl-3-azabicyclo[3.2.1]oct-8-yl)methyl]-4-cyclohexyl-N-methylbenzamide (Compound No. 85),
N-[(3-Benzyl-3-azabicyclo[3.2.1]oct-8-yl)methyl]biphenyl-4-carboxamide (Compound No. 86),
N-(3-Azabicyclo[3.2.1]oct-8-ylmethyl)-N-methyl-4′-(trifluoromethyl)biphenyl-2-carboxamide (Compound No. 87),
N-(3-Azabicyclo[3.2.1]oct-8-ylmethyl)-4-cyclohexylbenzamide (Compound No. 88),
N-(3-Azabicyclo[3.2.1]oct-8-ylmethyl)-4′-(trifluoromethyl)biphenyl-4-carboxamide (Compound No. 89),
N-(3-Azabicyclo[3.2.1]oct-8-ylmethyl)biphenyl-4-carboxamide (Compound No. 90),
N-(3-Azabicyclo[3.2.1]oct-8-ylmethyl)-2-biphenyl-4-yl-N-methylacetamide (Compound No. 91),
N-(3-Azabicyclo[3.2.1]oct-8-ylmethyl)-2-phenoxybenzamide (Compound No. 92),
N-(3-Azabicyclo[3.2.1]oct-8-ylmethyl)-3-benzyl-N-methylbenzamide (Compound No. 93),
   and pharmaceutically accepted salts, pharmaceutically acceptable solvates, enantiomers, diastereomers, polymorphs or N-oxides thereof.

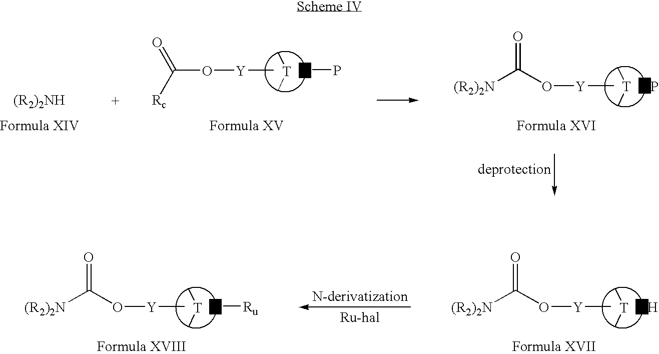

Compounds of Formulae XVII and XVIII may be prepared, for example, by the reaction sequence as shown in Scheme IV. In particular, compounds of Formula XIV (wherein R₂ is the same as defined earlier) can be condensed with compounds of Formula XV (wherein Y, T and P are the same as defined earlier; and R_{c} is heteroaryl or aryl) to form compounds of Formula XVI. Compounds of Formula XVI can be deprotected to form compounds of Formula XVII. Compounds of Formula XVII can be N-derivatized with compounds of Formula Ru-hal (wherein Rᵤ and hal are the same as defined earlier) to form compounds of Formula XVIII.

Compounds of Formula XIV can be condensed with compounds of Formula XV in the presence of one or more bases (for example, butyllithium, diisopropylamide, triethylamine or mixtures thereof). The reaction can also be carried out in one or more organic solvents (for example, tetrahydrofuran, dimethylformamide, diethylether, dioxane or mixtures thereof).

Compounds of Formula XVI (wherein P is aralkyl or —C(═O)Oaralkyl) can be deprotected to form compounds of Formula XVII in the presence of one or more deprotecting agents. Suitable deprotecting agents include, for example, palladium on carbon in presence of hydrogen gas or palladium on carbon with a source of hydrogen gas (for example, ammonium formate solution, cyclohexene or formic acid). The reaction can also be carried out in one or more organic solvents (for example, methanol, ethanol, propanol, isopropylalcohol or mixtures thereof).

Alternatively, compounds of Formula XVI (wherein P is —C(═O)Oaralkyl) can be deprotected in an alkaline solution. Suitable alkaline solutions comprise one or more bases (for example, potassium hydroxide, sodium hydroxide, lithium hydroxide or mixtures thereof) and one or more solvents (for example, methanol, ethanol propanol, diethylether, isopropylalcohol or mixtures thereof).

Compounds of Formula XVI (wherein P is —C(═O)OC(CH₃)₃ or —C(═O)OC(CH₃)₂CHBr₂) can be deprotected to form compounds of Formula XVII in an acidic solution (for example, hydrochloric acid solution in one or more solvents, e.g., methanol, ethanol, propanol, isopropylalcohol, ethyl acetate or ether) or trifluoroacetic acid in dichloromethane. Alternatively, the deprotection reaction can be carried out in the presence of one or more supernucleophiles (for example, lithium cobalt (I) phthalocyanine, zinc and acetic acid, cobalt phthalocyanine or mixtures thereof).

Compounds of Formula XVI (wherein P is —C(═O)OC(CH₃)₂CCl₃) can be deprotected in the presence of one or more supernucleophiles (for example, lithium cobalt (I) phthalocyanine, zinc and acetic acid, cobalt phthalocyanine or mixtures thereof).

Compounds of Formula XVII can be N-derivatized with compounds of Formula Ru-hal in the presence of one or more bases (for example, potassium carbonate, sodium carbonate, sodium bicarbonate or mixtures thereof). The reaction can also be carried out in one or more organic solvents (for example, acetonitrile, dichloromethane, chloroform, carbon tetrachloride or mixtures thereof).

Alternatively, compounds of Formula XVII can be N-derivatized by reductive amination in the presence of one or more reducing agents (for example, sodium cyanoborohydride, sodium triacetoxyborohydride or mixtures thereof). The reaction can also be carried out in one or more organic solvent (for example, acetonitrile or dichloromethane, tetrahydrofuran or mixtures thereof).

Exemplary compounds include, for example:
3-Azabicyclo[3.1.0]hex-6-ylmethyl benzyl(phenyl)carbamate (Compound No. 15),
(3-Benzyl-3-azabicyclo[3.1.0]hex-6-yl)methyl benzyl(phenyl)carbamate (Compound No. 41),
   and pharmaceutically accepted salts, pharmaceutically acceptable solvates, enantiomers, diastereomers, polymorphs or N-oxides thereof.

In the above schemes, where specific reagents or materials (e.g., bases, condensing agents, protecting groups, deprotecting agents, solvents, catalysts, temperatures, etc.) are mentioned, it is to be understood that other reagents or materials (e.g., bases, condensing agents, protecting groups, deprotecting agents, solvents, catalysts, temperatures, etc.) known to those skilled in the art may be used. Similarly, reaction conditions (e.g., temperature and duration) may be adjusted according to the desired needs.

Suitable salts of the compounds described herein can be prepared to solubilize such compounds in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are included as substituents in the compounds described herein, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

The compounds described herein can be produced and formulated as their enantiomers, diastereomers, N-oxides, polymorphs, solvates and pharmaceutically acceptable salts, as well as metabolites having the same type of activity. Provided are pharmaceutical compositions comprising the compounds described herein or metabolites, enantiomers, diastereomers, N-oxides, polymorphs, solvates or pharmaceutically acceptable salts thereof, in combination with one or more pharmaceutically acceptable carriers and one or more optional excipient.

Compounds described herein or pharmaceutically acceptable salts, pharmaceutically acceptable solvates, stereoisomers, tautomers, racemates, prodrugs, metabolites, polymorphs or N-oxides thereof, may be advantageously used in combination with one or more other therapeutic agents. Examples of other therapeutic agents include, but are not limited to, corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or mixtures thereof.

Any suitable route of administration may be employed for providing the patient with an effective dosage of one or more compounds described herein according to the methods of the present invention. For example, oral, intraoral, rectal, parenteral, epicutaneous, transdermal, subcutaneous, intramuscular, intranasal, sublingual, buccal, intradural, intraocular, intrarespiratory, or nasal inhalation and like forms of administration may be employed. Oral administration is generally preferred.

In one example, compounds described herein can be administered by inhalation or insufflation. Compounds described herein for inhalation or insufflation include solutions and suspensions in pharmaceutically acceptable, aqueous or organic solvents or mixtures thereof, and powders. Liquid or solid compositions may contain suitable pharmaceutically acceptable excipients. The compositions can be administered by the nasal respiratory route for local or systemic effect. Compositions can be nebulized by use of inert gases. Nebulized solutions may be breathed directly from the nebulizing device or the nebulizing device can be attached to a face masks tent or intermittent positive pressure breathing machine. Solutions, suspensions or powder compositions can be administered nasally from devices, which deliver the formulation in an appropriate manner.

Alternatively, compositions can be administered orally, rectally, parenterally (intravenously, intramuscularly or subcutaneously), intracistemally, intravaginally, intraperitoneally or topically.

Solid dosage forms for oral administration may be presented in discrete units, for example, capsules, cachets, lozenges, tablets, pills, powders, dragees or granules, each containing a predetermined amount of the active compound. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms can be prepared with coatings and shells, such as enteric coatings and others well known in this art. They may contain opacifying agents, and can also be of such composition that they release the active compound or compounds in a certain part of the intestinal tract in a delayed manner. Examples of embedding compositions which can be used are polymeric substances and waxes.

The active compounds can also be in micro-encapsulated form, if appropriate, with one or more of the above mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, for example, wetting agents, emulsifying and suspending agents, sweetening, flavoring and perfuming agents, colorants or dyes.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth or mixtures of these substances, and the like.

Dosage forms for topical administration of a compound of this invention include powders, sprays, inhalants, ointments, creams, salves, jellies, lotions, pastes, gels, aerosols or oils. The active component is admixed under sterile conditions with a pharmaceutically acceptable carrier and any needed preservatives, buffers or propellants as may be required. Ophthalmic formulations, eye ointments, powders and solutions are also contemplated as being within the scope of this invention.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and non-aqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monosterate and gelatin.

Suppositories for rectal administration of compounds described herein can be prepared by mixing the drug with a suitable nonirritating excipient such as cocoa butter and polyethylene glycols or a suppository wax, which are solid at ordinary temperatures but liquid at body temperature and which therefore melt in the rectum or vaginal cavity and release the drug.

If desired, and for more effective distribution, compounds described herein can be incorporated into slow release or targeted delivery systems such as polymer matrices, liposomes, and microspheres. They may be sterilized, for example, by filtration through a bacteria-retaining filter or by incorporating sterilizing agents in the form of sterile solid compositions, which can be dissolved in sterile water or some other sterile injectable medium immediately before use.

Actual dosage levels of active ingredients in the compositions and spacing of individual dosages may be varied so as to obtain an amount of active ingredient that is effective to obtain a desired therapeutic response for a particular composition and method of administration. It will be understood, however, that the specific dose level for any particular patient will depend upon a variety of factors including the compound chosen, the body weight, general health, sex, diet, route of administration, the desired duration of treatment, rates of absorption and excretion, combination with other drugs and the severity of the particular disease being treated and is ultimately at the discretion of the physician.

The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### Example 1

### Synthetic procedure for 2-(2-thienyl)benzoic acid

### Step-I: Methyl-2-{[trifluoro methyl)sulfonyl]oxy}benzoate

Pyridine (59.2 mmol) was added dropwise to a solution of methyl salicylate (Commercially available) (3 g, 19.73 mmol) in dry dichloromethane (50 mL) under nitrogen atmosphere and the reaction mixture was cooled to −10° C. Triflic anhydride (59.2 mmol) was added dropwise to the reaction mixture and stirred at the same temperature for 1 hour. The reaction mixture was diluted with water and stirred for 15 minutes. The organic layer thus separated was washed sequentially with hydrochloric acid (0.2 N), water and brine. The residue thus obtained was purified by column chromatography using 4% ethyl acetate in hexane as an eluent to furnish the title compound. Yield: 2.7 g

### Step-II: Methyl 2-(2-thienyl)benzoate

A mixture of the compound obtained from step-I above (500 mg; 198 mmol), thiophene-2-boronic acid (2.18 mmol), tetrakis(triphenyl phosphine) palladium (0.1 mmol) and potassium phosphate (7.86 mmol) in dry dimethylformamide (10 mL) was refluxed under nitrogen atmosphere for 16 hours. The reaction mixture was then poured into water and extracted with ethyl acetate. The resulting organic layer was washed with sodium bicarbonate solution and brine, filtered and concentrated under reduced pressure. The residue thus obtained was purified by column chromatography using ethyl acetate in hexane as the eluent to furnish the title compound. Yield: 200 mg.

### Step-III: 2-(2-Thienyl)benzoic acid

Aqueous potassium hydroxide solution (11.45 mmol) was added to a suspension of the compound obtained from step-II above (500 mg, 2.29 mmol) in methanol and stirred at room temperature for 6 hours. The reaction mixture was concentrated under reduced pressure and the residue thus obtained was diluted with water, acidified with concentrated hydrochloric acid and stirred. The acidified mixture was then extracted with ethyl acetate, washed with water and brine, dried over anhydrous sodium sulphate and concentrated under reduced pressure to furnish the title compound. Yield: 420 mg.

¹H NMR: δ 7.90-7.88 (1H, m), 7.52-7.35 (4H, m), 7.09-7.06 (2H, m).

IR: 1694.3 cm⁻¹.

### Example 2

### Synthesis of 2′,4′-difluorobiphenyl-2-carboxylic acid

### Step-I: Methyl 2′,4′-difluorobiphenyl-2-carboxylate

2,4 difluorophenyl boronic acid (716 mg, 4.539 mmol), tetrakis-triphenylphosphine palladium (238 mg, 0.206 mmol) and potassium phosphate (3.48 g, 16.33 mmol) was added to a solution of the compound methyl-2-{[trifluoromethyl)sulfonyl]oxy}benzoate (1.04 g, 4.126 mmol) in dry dimethylformamide (20 mL), and the reaction mixture was refluxed under nitrogen atmosphere for 16 hours. The reaction mixture was filtered and the filtrate was poured into water and extracted with ethyl acetate. The organic layer was washed with sodium bicarbonate and brine, dried over anhydrous sodium sulphate and concentrated under reduced pressure. The residue thus obtained was purified by column chromatography using 2% ethyl acetate in hexane as eluent to furnish the title compound.

Yield: 500 mg.

### Step-II: Synthesis of 2′,4′-difluorobiphenyl-2-carboxylic acid

Aqueous potassium hydroxide (16.13 mmol, 903 mg, in 5 mL water) was added to a solution of the compound obtained from step-I above (800 mg) in methanol (20 mL) and the reaction mixture was stirred for 6 hours at room temperature. The reaction mixture was concentrated under reduced pressure and the residue thus obtained was diluted with water, acidified with concentrated hydrochloric acid and stirred. The organic layer was separated, washed with water and brine, dried over anhydrous sodium sulphate and concentrated under reduced pressure to furnish the title compound. Yield: 620 mg.

¹H NMR (CDCl₃) δ: 8.08 (1H, d), 7.62 (1H, m), 7.48 (m, 1H), 7.32 (m, 2H), 6.84-6.96 (m, 2H).

IR (DCM): 1695.8 cm⁻¹.

### Example 3

### Synthesis of 2-benzylbenzoic acid

### Step-I: Synthesis of 2-benzylbenzoic acid

Solid aluminum chloride (9.9 g, 74.3 mmol) was added slowly in portions to a suspension of phthalic anhydride (5 g, 33.75 mmol) in benzene (20 mL). The reaction mixture was warmed to 50° C.-60° C. for 3-4 minutes followed by the addition of remaining aluminum chloride (3 g). The reaction mixture was warmed followed by refluxing until hydrogen chloride gas ceased to evolve. The reaction mixture was cooled to room temperature and poured slowly into a solution of crushed ice and concentrated hydrochloric acid (20 mL). The reaction mixture was extracted with ethyl acetate, washed with water and brine, dried over anhydrous sodium sulphate and concentrated under reduced pressure to furnish the title compound. Yield: 8.2 g.

### Step-II: Synthesis of 2-benzyl benzoic acid

Palladium on carbon (400 mg, 20% w/w) and anhydrous ammonium formate (1.6 g, 25.6 mmol) was added to the solution of the compound obtained from step-I above (1 g, 4.42 mmol) in glacial acetic acid (15 mL). The reaction mixture was stirred at 110° C. for 1 hour and subsequently diluted with chloroform. The reaction mixture was filtered over a celite pad and washed with chloroform. The filtrate was concentrated under reduced pressure and the residue thus obtained was diluted with water. The reaction mixture was extracted with dichloromethane. The organic layer was separated, washed with water and brine, dried over anhydrous sodium sulphate and concentrated to furnish the title compound. Yield: 795 mg.

¹H NMR (CDCl₃) δ: 7.87 (1H, d), 7.42 (1H, m), 7.1-7.29 (7H, m), 4.37 (2H, s).

IR (KBr): 1693.4 cm⁻¹.

### Example 4

### Synthesis of (3-benzyl-3-azabicyclo[3.2.1]oct-8-yl)methanol

### Step-I: 3-Benzyl-3-aza-bicyclo[3.2.1]octane-8-one

A solution of cyclopentanone (1180.0 mmol), paraformaldehyde (3540.0 mmol) and glacial acetic acid (1180.0 mmol) in methanol (600 mL) was refluxed for 3 hours. A solution of benzylamine (118.0 mmol) in methanol (200 mL) was added dropwise to the resulting reaction mixture. The reaction mixture was refluxed for 1 hour and subsequently stirred at room temperature overnight. The reaction mixture was concentrated under reduced pressure and the residue thus obtained was diluted with ethyl acetate followed by the addition of sodium metabisulphite (104.6 g). The reaction mixture was stirred for 1 hour and the aqueous layer was separated, cooled under ice and neutralized with sodium carbonate solution. The reaction mixture was extracted with ethyl acetate, washed with water and brine, dried over anhydrous sodium sulphate and concentrated under reduced pressure. The residue thus obtained was purified by column chromatography using 5% ethyl acetate in hexane as eluent to furnish the title compound. Yield: 37.5 g.

### Step-II: 3-benzyl-3-azabicyclo[3.2.1]octane-8-carbaldehyde

N-butyl lithium (13.7 mL, 34.88 mmol) was added to a solution of (methoxymethyl)(triphenyl)phosphonium chloride (11.9 g, 34.88 mmol) in tetrahydrofuran (50 mL) that was cooled to −50° C. The reaction mixture was stirred at −25° C. for 30 minutes followed by the dropwise addition of a solution of the compound obtained from step-I above (5 g, 23.25 mmol) in tetrahydrofuran (10 mL) at the same temperature. The resulting reaction mixture was stirred at room temperature overnight. The reaction mixture was concentrated under reduced pressure and the residue thus obtained washed with hexane, dried under reduced pressure and the residue thus obtained was diluted with tetrahydrofuran followed by the addition of aqueous hydrochloric acid (20%, 30 mL). The reaction mixture was stirred at room temperature for 5 hours and then the organic layer was evaporated under reduced pressure. The aqueous layer was basified with aqueous potassium hydroxide and extracted with ethyl acetate. The organic layer was concentrated under reduced pressure and the residue thus obtained was diluted with saturated solution of sodium metabisulphite. The organic layer was separated and neutralized with sodium carbonate. The reaction mixture was extracted with ethyl acetate, the organic layer was concentrated under reduced pressure and the residue thus obtained was treated with methanol-water-sodium hydroxide. The solution was stirred at room temperature for 3 days. The reaction mixture was concentrated under reduced pressure and the residue thus obtained was diluted with water. The reaction mixture was extracted with ethyl acetate. The organic layer was washed with water and brine, dried under reduced pressure, filtered and concentrated under reduced pressure to furnish the title compound. Yield: 2.2 g.

¹H NMR (CDCl₃) δ: 9.62 (s, 1H), 7.32-7.22 (m, 5H), 3.51 (s, 2H), 2.79-2.75 (m, 2H), 2.51 (bs, 2H), 2.25 (s, 1H), 2.13-2.05 (m, 2H), 1.79-1.77 (m, 2H), 1.60-1.57 (m, 2H).

### Step-III: (3-benzyl-3-azabicyclo[3.2.1]oct-8-yl)methanol

Sodium borohydride (0.545 g, 14.41 mmol) was added to a solution of the compound obtained from step-II above (2.2 g, 9.606 mmol) in methanol cooled in an ice-bath, and the reaction mixture was stirred for 2 hours at the same temperature. The reaction mixture was concentrated under reduced pressure and the residue thus obtained was diluted with ethyl acetate, washed with water and brine, dried over anhydrous sodium sulphate and concentrated under reduced pressure to furnish the title compound. Yield: 1.8 g.

¹H NMR (CDCl₃) δ: 7.33-7.19 (m, 5H), 3.48 (s, 2H), 3.43-3.41 (d, 2H, J=8 Hz), 2.73-2.69 (m, 2H), 2.09-2.05 (m, 4H), 1.74-1.42 (m, 5H).

### Example 5

### Synthesis of (3-benzyl-3-azabicyclo[3.1.0]hex-6-yl)methanol

### Step a: Ethyl 5-benzyl-4,6-dioxo-1,3a,4,5,6,6a-hexahydropyrrolo[3,4-c]pyrazole-3-carboxylate

N-benzylmaleimide (64 g) was added to a solution of ethyl diazoacetate (1 eq.) in dichloromethane (10 mL) and the reaction mixture was stirred at room temperature for five days. The reaction mixture was cooled in an ice-bath and stirred for about 2 hours. The crystals thus separated were filtered over a celite pad and washed with hexane to furnish the title compound.

### Step b: Ethyl 3-benzyl-2,4-dioxo-3-azabicyclo[3.1.0]hexane-6-carboxylate

The compound obtained from Step a above (20 g) was slowly added to a beaker which was melted at 190° C. followed by the slow addition of an additional amount of the compound obtained from step a above (180 g). The reaction mixture was stirred at same temperature for 30 minutes. The compound was cooled to room temperature and diluted with ether. The solution was cooled in dry ice acetone bath for about 2 hours. The resulting reaction mixture was subsequently brought to room temperature and filtered over a celite pad to furnish the title compound.

### Step c: (3-Benzyl-3-azabicyclo[3.1.0]hex-6-yl)methanol

A solution of lithium aluminum hydride (1.114 g) in dry tetrahydrofuran (10 mL) was added to a precooled solution of the compound obtained from step b above (2 g) in dry tetrahydrofuran (10 mL) at −78° C. The reaction mixture was brought to room temperature and then subsequently refluxed overnight. The reaction mixture was cooled in dry ice-acetone bath and the reaction mixture subsequently was quenched by addition of a saturated solution of sodium sulphate in water. The reaction mixture was filtered over a celite pad and the filtrate was dried over anhydrous sodium sulphate and concentrated under reduced pressure to furnish the title compound. Yield: 1.56 g.

### Example 6

### Synthesis of 1-(3-benzyl-3-azabicyclo[3.1.0]hex-6-yl)methanamine

### Step a: 3-Benzyl-3-azabicyclo[3.1.0]hexane-6-carbaldehyde

Dimethylsulphoxide (19.24 g, 17.5 mL) dilute in dichloromethane (500 mL) was added to a precooled solution of oxalyl chloride (15.6 g, 10.72 mL) in dichloromethane (250 mL) at −78° C. under nitrogen atmosphere. The reaction mixture was stirred for 60 minutes followed by the dropwise addition of a solution of (3-benzyl-3-azabicyclo[3.1.0]hex-6-yl)methanol (10.0 g) in dichloromethane (500 mL). The reaction mixture was stirred at the same temperature for about 2 hours followed by the addition of triethylamine (68.8 mL). The resulting reaction mixture was subsequently stirred at the same temperature followed by stirring at room temperature overnight. The reaction mixture was concentrated under reduced pressure and the residue thus obtained was partitioned between ethyl acetate and water. The organic layer was separated, washed with water and brine, dried over anhydrous sodium sulphate and concentrated under reduced pressure to furnish the title compound.

### Step b: 3-Benzyl-3-azabicyclo[3.1.0]hexane-6-carbaldehyde oxime

Sodium acetate (62.6 g) and hydroxyl amine hydrochloride (95.13 g) were added to a solution of the compound obtained from step a above (43 g) in ethanol (1000 mL). The reaction mixture was stirred 11 hours and excess solvent was evaporated under reduced pressure. The residue thus obtained was partitioned between dichloromethane and potassium carbonate. The combined organic layer was dried over anhydrous sodium sulphate and concentrated under reduced pressure to furnish the title compound.

### Step c: 1-(3-Benzyl-3-azabicyclo[3.1.0]hex-6-yl)methanamine

Lithium aluminum hydride (0.8045 g) was added to a solution of the compound obtained from step b above (1.01 g) in tetrahydrofuran (50 mL) and the reaction mixture was refluxed for 12 hours. Water and saturated solution of ammonium chloride were added to the resulting reaction mixture. The reaction mixture was filtered over a celite pad and concentrated under reduced pressure to furnish the title compound.

### Example 7

### Synthesis of tert-butyl 8-(hydroxymethyl)-3-azabicyclo[3.2.1]octane-3-carboxylate

### Step-I: 3-Azabicyclo[3.2.1]oct-8-ylmethanol

Palladium on carbon was added to a solution of the compound 3-benzyl-3-azabicyclo[3.2.1]oct-8-yl)methanol (1 g) in methanol (10 mL) and the reaction mixture was stirred under hydrogen atmosphere overnight. The reaction mixture was filtered over a celite pad and washed with methanol. The filtrate was concentrated under reduced pressure to furnish the title compound. Yield: 820 mg.

### Step-II: Tert-butyl 8-(hydroxymethyl)-3-azabicyclo[3.2.1]octane-3-carboxylate

Triethylamine (6.98 mmol) was added to a solution of the compound obtained from step-I above (820 mg, 5.82 mmol) in dichloromethane (10 mL) followed by the addition of ditert-butoxycarbonyl anhydride (6.4 mmol). The reaction mixture was stirred at room temperature overnight and then washed with sodium bicarbonate solution. The organic layer was separated, washed with water and brine, dried over anhydrous sodium sulphate and concentrated under reduced pressure to furnish the title compound.

¹H NMR (CDCl₃) δ: 3.95 (d, 1H), 3.79 (d, 1H), 3.32 (d, 2H), 2.84 (dd, 2H), 2.12 (d, 2H), 1.84 (t, 1H), 1.67-1.61 (m, 4H), 1.48 (s, 9H).

Mass (m/z): 242.3 (M⁺+1).

### Example 8

### Synthesis of N-benzyl-4-fluoroaniline

Sodium triacetoxyborohydride (5.97 g, 28.2 mmol) was added under nitrogen atmosphere to a solution of para-fluoro benzaldehyde (1 g, 9.4 mmol) and 4-fluoroaniline (1.15 g, 10.34 mmol) in dichloroethane (30 mL) and the reaction mixture was stirred at room temperature for 18 hours. The solvent was evaporated off under reduced pressure and the residue thus obtained was partitioned between ethyl acetate and 5% aqueous potassium hydroxide solution. The aqueous layer was extracted with ethyl acetate and the organic layer was washed with water and brine, dried over anhydrous sodium sulphate and concentrated under reduced pressure. The residue thus obtained was purified by column chromatography using 2% ethyl acetate in hexane as the eluent to furnish the title compound. Yield: 1.45 g.

### Example 9

### Synthesis of (3-benzyl-3-azabicyclo[3.1.0]hex-6-yl)methyl biphenyl-2-ylcarbamate (Compound No. 1)

Diphenyl phosphonic azide (2.65 mmol) and triethylamine (2.77 mmol) were added to the solution of biphenyl-2 carboxylic acid (Commercially available) (500 mg, 2.52) mmol) in dry toluene (˜15 mL). The reaction mixture was stirred at 60° C. for 1.5 hours and then (3-benzyl-3-azabicyclo[3.1.0]hex-6-yl)methanol (588 mg, 2.89 mmol) was added. The reaction mixture was refluxed for six hours followed by stirring at room temperature overnight. The reaction mixture was quenched with water and extracted with ethyl acetate. The organic layer was separated, washed with water and brine, dried over anhydrous sodium sulphate and concentrated under reduced pressure. The residue thus obtained was purified by column chromatography using 10% ethyl acetate in hexane as eluent to furnish the title compound. Yield: 700 mg.

¹H NMR (CDCl₃) δ: 7.48-7.19 (14H, m), 6.63 (1H, s), 3.94-3.91 (2H, d), 3.57 (2H, s), 2.98-2.95 (2H, m), 2.35-2.32 (2H, m), 1.56-1.11 (3H, m).

Analogs of (3-benzyl-3-azabicyclo[3.1.0]hex-6-yl)methyl biphenyl-2-ylcarbamate (Compound No. 1) described below can be prepared by condensing an appropriate corresponding acid with an appropriate corresponding amine or alcohol, respectively, as applicable in each case.

### N-[(3-benzyl-3-azabicyclo[3.1.0]hex-6-yl)methyl]-N′-biphenyl-2-ylurea

### Compound No. 2

¹H NMR (CDCl₃) δ: 7.44-7.11 (14H, m), 6.11 (1H, s), 4.65 (1H, s), 3.55 (2H, s), 2.99-2.89 (4H, m), 2.32-2.29 (2H, m), 1.21-1.14 (3H, m).

Mass (m/z): 398 (M⁺+1).

IR (DCM): 1646 cm⁻¹.

### 3-Benzyl-3-azabicyclo[3.2.1]oct-8-yl biphenyl-2-ylcarbamate

### Compound No. 4

¹H NMR (CDCl₃) δ: 7.51-7.15 (14H, m), 6.63 (1H, s), 4.73-4.70 (1H, t), 3.45 (2H, s), 2.46-2.43 (4H, m), 2.42-2.38 (2H, m), 1.86-1.80 (2H, m), 1.71-1.66 (2H, m).

Mass (m/z): 413 (M⁺+1).

IR (DCM): 1726 cm⁻¹

### 2-Benzyl-2-azabicyclo[2.2.1]hept-7-yl biphenyl-2-ylcarbamate

### Compound No. 6

¹H NMR (CDCl₃) δ: 7.47-7.14 (14H, m), 6.54 (1H, s), 5.0 (1H, s), 3.71 (2H, s), 3.23 (1H, s), 2.97 (1H, s), 2.93-2.91 (2H, m), 1.31-1.25 (4H, m).

Mass (m/z): 399 (M⁺+1).

### (3-Benzyl-3-azabicyclo[3.2.1]oct-8-yl)methyl biphenyl-2-ylcarbamate

### Compound No. 7

¹H NMR (CDCl₃) δ: 7.38-7.20 (14H, m), 6.60 (1H, s), 3.93-3.91 (2H, d), 3.47 (2H, s), 2.74-2.67 (2H, m), 2.09-2.01 (4H, m), 1.65-1.56 (3H, m), 1.32-1.28 (2H, m).

Mass (m/z): 427 (M⁺+1).

### (3-Benzyl-3-azabicyclo[3.1.0]hex-1-yl)methylbiphenyl-2-ylcarbamate

### Compound No. 8

¹H NMR (CDCl₃) δ: 7.76-7.20 (14H, m), 6.59 (1H, s), 4.29-4.26 (1H, d), 4.10-4.07 (1H, d), 3.60-3.58 (2H, m), 2.99-2.92 (2H, m), 2.39-2.32 (2H, m), 1.32-1.30 (2H, m), 1.16-1.14 (1H, m).

Mass (m/z): 399 (M⁺+1)

### (3-Benzyl-3-azabicyclo[3.1.0]hex-6-yl)methyl[2-(2-thienyl)phenyl]carbamate

### Compound No. 9

¹H NMR (CDCl₃) δ: 7.42-7.09 (12H, m), 6.97 (1H, s), 3.97-3.95 (2H, d), 3.57 (2H, s), 2.99-2.97 (2H, m), 2.36-2.33 (2H, m), 1.36 (1H, m), 1.28-1.24 (2H, m).

Mass (m/z): 405 (M⁺+1)

### (3-Benzyl-3-azabicyclo[3.1.0]hex-6-yl)methyl (2′,4′-difluorobiphenyl-2-yl)carbamate

### Compound No. 11

¹H NMR (CDCl₃) δ: 8.05 (1H, d), 7.4 (1H, m), 7.31-7.13 (8H, m), 7.02-6.93 (2H, m), 6.33 (1H, s), 3.92 (2H, d), 3.57 (2H, s), 2.97 (2H, d), 2.34 (2H, d), 1.64 (1H, s), 1.30-1.25 (2H, s)

Mass (m/z): 435.3 (M⁺+1).

### (3-Benzyl-3-azabicyclo[3.1.0]hex-1-yl)methyl (2′,4′-difluorobiphenyl-2-yl)carbamate

### Compound No. 12

¹H NMR (CDCl₃) δ: 7.97 (s, 1H), 6.95-7.40 (m, 11H), 6.30 (1H, s), 4.28 (1H, d), 4.08 (1H, d), 3.60 (dd, 2H), 2.96 (dd, 2H), 2.32 (dd, 2H), 1.15 (s, 1H), 0.9 (m, 1H), 0.53 (1H, m).

### 3-Benzyl-3-azabicyclo[3.1.0]hex-6-yl)methyl(2′,4′-dimethoxybiphenyl-2-yl)carbamate

### Compound No. 13

¹H NMR (CDCl₃) δ: 7.98 (1H, s), 7.11-7.34 (9H, m), 6.62 (1H, s), 6.59 (m, 2H), 3.92 (d, 2H), 3.87 (s, 3H), 3.78 (s, 3H), 3.57 (s, 2H), 2.96 (d, 2H), 2.34 (d, 2H), 1.34-1.28 (3H, m).

Mass (m/z): 459.2 (M⁺+1).

### (3-Benzyl-3-azabicyclo[3.1.0]hex-6-yl)methyl[2-(1,3-benzodioxol-5-yl)phenyl]carbamate

### Compound No. 19

¹H NMR (CDCl₃) δ: 8.13 (d, 1H), 7.35-7.10 (m, 7H), 6.92 (d, 1H), 6.80 (m, 2H), 6.67 (s, 1H), 6.03 (s, 2H), 3.94 (d, 2H), 3.58 (s, 2H), 2.98 (d, 2H), 2.35 (d, 2H), 2.05 (s, 1H), 1.62 (s, 1H), 1.36 (s, 2H).

Mass (m/z): 443.1 (M⁺+1).

### (3-Benzyl-3-azabicyclo[3.2.1]oct-8-yl)methyl(2-benzylphenyl)carbamate

### Compound No. 20

¹H NMR (CDCl₃) δ: 7.74 (s, 1H), 7.07-7.33 (m, 13H), 6.29 (s, 1H), 3.97 (s, 2H), 3.90 (2H, d), 3.48 (2H, s), 2.70 (2H, d), 2.09 (2H, d), 2.00 (2H, s).

Mass (m/z): 441.1 (M⁺+1).

### (3-benzyl-3-azabicyclo[3.2.1]oct-8-yl)methyl(2-benzoylphenyl)carbamate

### Compound No. 28

¹H NMR (CDCl₃) δ: 10.26 (1H, s), 8.44-8.42 (1H, m), 7.70-7.02 (13H, m), 3.98-3.96 (2H, d), 3.48 (2H, s), 2.72-2.69 (2H, m), 2.12-2.04 (4H, m), 1.90-1.86 (1H, m), 1.79-1.73 (4H, m).

### (3-Benzyl-3-azabicyclo[3.2.1]oct-8-yl)methyl[2-(4-methylbenzyl)phenyl]carbamate

### Compound No. 29

¹H NMR (CDCl₃) δ: 7.33-7.10 (13H, m), 6.31 (1H, s), 3.91-3.88 (4H, m), 3.48 (2H, s), 2.71-2.68 (2H, m), 2.30-2.28 (4H, m), 1.99 (1H, m), 1.82-1.75 (4H, m).

### (3-Benzyl-3-azabicyclo[3.1.0]hex-1-yl)methyl[2-(2-thienyl)phenyl]carbamate

### Compound No. 39

¹H NMR (CDCl₃) δ: 7.41-7.10 (12H, m), 6.86 (1H, s), 4.32-4.29 (1H, d), 4.14-4.11 (1H, d), 3.61-3.59 (2H, m), 3.02-2.93 (2H, m), 2.37-2.35 (2H, m), 1.16 (1H, m), 0.98-0.88 (2H, m)

Mass (m/z): 405 (M⁺+1).

### (3-Benzyl-3-azabicyclo[3.2.1]oct-8-yl)methyl (2-ethoxyphenyl)carbamate

### Compound No. 58

¹H NMR (CDCl₃) δ: 8.08 (1H, bs), 7.34-7.22 (5H, m), 6.94 (2H, m), 6.84 (1H, m), 4.08 (2H, q, 7.2 Hz), 3.97 (2H, d, 7.6 Hz), 3.53 (2H, s), 2.76 (2H, bm), 2.17-2.10 (4H, m), 1.88-1.69 (6H, m), 1.44 (3H, t, 7.2 Hz).

Mass (m/z): 395 (M⁺+1).

### (3-Benzyl-3-azabicyclo[3.2.1]oct-8-yl)methyl(2-hydroxy-3-methoxyphenyl)carbamate

### Compound No. 59

¹H NMR (CDCl₃) δ: 7.37-6.48 (8H, m), 3.94 (2H, d, 8 Hz), 3.70 (3H, s), 3.51 (2H, bs), 2.74 (2H, bs), 2.06 (4H, bs), 1.84 (1H, m).

### (3-Benzyl-3-azabicyclo[3.2.1]oct-8-yl)methyl(3,4-dimethoxyphenyl)carbamate

(Compound NO. 60)

¹H NMR (CDCl₃) δ: 7.32-7.19 (6H, m), 6.79-6.73 (2H, m), 6.66 (1H, bs), 3.94 (2H, bs), 3.86 (3H, s), 3.84 (3H, s), 3.57 (2H, s), 2.81 (2H, bd), 2.18 (2H, d, 10 Hz), 2.09 (2H, bs), 1.88-1.82 (3H, m), 1.69 (2H, bs).

Mass (m/z): 411 (M⁺+1).

### (3-Benzyl-3-azabicyclo[3.2.1]oct-8-yl)methyl biphenyl-2-ylcarbamate

### Compound No. 61

¹H NMR (CDCl₃) δ: 8.07 (1H, bs), 7.73 (2H, d, 8 Hz), 7.50 (2H, d, 8 Hz), 7.40-7.16 (8H, m), 6.43 (1H, s), 3.92 (2H, d, 8 Hz), 3.52 (2H, s), 2.74 (2H, bd), 2.13-1.64 (11H, m).

Mass (m/z): 495 (M⁺+1).

### (3-Benzyl-3-azabicyclo[3.2.1]oct-8-yl)methyl(4-phenoxyphenyl)carbamate

### Compound No. 62

¹H NMR (CDCl₃) δ: 7.33-7.24 (9H, m), 7.09-6.96 (5H, m), 6.55 (1H, s), 3.97 (2H, d, 8 Hz), 3.52 (2H, bs), 2.75 (2H, bs), 2.09 (4H, bs), 1.89-1.25 (5H, bm).

Mass (m/z): 443 (M⁺+1).

### (3-Benzyl-3-azabicyclo[3.2.1]oct-8-yl)methyl biphenyl-4-ylcarbamate (Compound No. 63)

¹H NMR (CDCl₃) δ: 7.57-7.26 (13H, m), 6.67 (1H, s), 3.98 (2H, d, 8 Hz), 3.62 (2H, bs), 2.85 (2H, bd), 2.24 (2H, d, 10.4 Hz), 2.13 (2H, s), 1.89 (3H, bm), 1.72 (2H, bs).

Mass (m/z): 427 (M⁺+1).

### (3-Benzyl-3-azabicyclo[3.2.1]oct-8-yl)methyl[2-(4-methoxybenzyl)phenyl]carbamate

### Compound No. 64

¹H NMR (CDCl₃) δ: 7.33-6.81 (13H, m), 6.5 (1H, bs), 3.90 (2H, bs), 3.76 (3H, s), 3.5 (2H, bs), 2.7 (2H, bs), 2.02 (4H, bs), 1.83-1.25 (7H, m).

Mass (m/z): 471 (M⁺+1).

### (3-Benzyl-3-azabicyclo[3.2.1]oct-8-yl)methyl[2-(3-methoxybenzoyl)phenyl]carbamate

### Compound No. 65

¹H NMR (CDCl₃) δ: 8.36 (1H, d, 8 Hz), 7.73 (2H, d, 8 Hz), 7.54 (2H, t, 8 Hz), 7.32 (5H, bs), 7.06-6.95 (3H, m), 3.95 (2H, d, 8 Hz), 3.90 (5H, s), 3.59 (2H, bs), 2.8 (2H, bs), 2.13 (4H, bs), 1.89-1.25 (3H, m).

Mass (m/z): 485 (M⁺+1).

### (3-Benzyl-3-azabicyclo[3.2.1]oct-8-yl)methyl[2-(4-methylbenzoyl)phenyl]carbamate

### Compound No. 67

¹H NMR (CDCl₃) δ: 8.40 (1H, d, 8 Hz), 7.63 (2H, d, 8H), 7.55 (2H, m), 7.33-7.00 (8H, m), 3.96 (2H, d, 8 Hz), 3.48 (2H, s), 2.70 (2H, m), 2.43 (3H, s), 2.10 (4H, d, 12 Hz).

Mass (m/z): 469 (M⁺+1).

### Example 10

### Synthesis of 3-azabicyclo[3.1.0]hex-6-ylmethyl biphenyl-2-ylcarbamate (Compound No. 46)

Palladium on carbon (10%) and ammonium formate were added to a solution of Compound No. 1 (440 mg) in methanol (35 mL). The reaction mixture was then refluxed for 1 hour and allowed to come to room temperature. The reaction mixture was filtered through a celite pad and washed with methanol. The filtrate was concentrated under reduced pressure and the residue thus obtained was dissolved in dichloromethane followed by the addition of water. The reaction mixture was basified with aqueous sodium hydroxide solution (10%). The organic layer was washed with water and brine, dried over anhydrous sodium sulphate and concentrated under reduced pressure to furnish the title compound. Yield: 285 mg.

¹H NMR (CDCl₃) δ: 7.49-7.12 (9H, m), 6.65 (1H, s), 3.94-3.91 (2H, m), 3.09-2.86 (4H, m), 1.01-0.95 (3H, m).

Mass (m/z): 309 (M⁺+1).

Analogs of 3-azabicyclo[3.1.0]hex-6-ylmethyl biphenyl-2-ylcarbamate (Compound No. 46) described below can be prepared by deprotecting appropriate corresponding benzylated compound.

### 3-Azabicyclo[3.2.1]oct-8-yl biphenyl-2-ylcarbamate

### Compound No. 5

¹H NMR (CDCl₃) δ: 7.52-7.12 (9H, m), 6.76 (1H, s), 4.85-4.81 (1H, t), 3.13-3.09 (2H, m), 2.51-2.47 (2H, m), 1.84-1.81 (2H, m), 1.71-1.43 (4H, m).

Mass (m/z): 323 (M⁺+1).

### 3-Aabicyclo[3.1.0]hex-6-ylmethyl[2-(2-thienyl)phenyl]carbamate

### Compound No. 10

¹H NMR (CDCl₃) δ: 7.44-7.09 (7H, m), 6.09 (1H, s), 4.05-4.03 (2H, d), 3.02-2.84 (4H, m), 1.69 (1H, m), 0.99-0.85 (2H, m).

### 3-Azabicyclo[3.1.0]hex-6-ylmethyl (2′,4′-difluorobiphenyl-2-yl)carbamate

### Compound No. 17

¹H NMR (CDCl₃) δ: 7.96 (1H, s), 7.04-7.44 (1H, m), 7.19-7.317 (4H, m), 7.028-7.066 (2H, m), 6.401 (1H, s), 4.20-4.22 (2H, d), 3.116-3.446 (4H, m), 1.69 (1H, m), 1.18 (1H, m), 0.93 (1H, m).

Mass (m/z): 345 (M⁺+1).

### 3-Azabicyclo[3.1.0]hex-6-ylmethyl (2′,4′-dimethoxybiphenyl-2-yl)carbamate

### Compound No. 18

¹H NMR (CDCl₃) δ: 7.95 (s, 1H), 7.35 (m, 1H), 7.17 (2H, m), 6.78 (1H, s), 6.63 (2H, d), 3.98 (2H, s), 3.88 (3H, s), 3.80 (3H, s), 3.38 (2H, d), 3.27 (2H, d), 1.41 (1H, m), 1.70 (2H, s).

### 3-Azabicyclo[3.2.1]oct-8-ylmethyl (2-benzylphenyl)carbamate

### Compound No. 22

¹H NMR (CDCl₃) δ: 7.74 (1H, s), 7.08-7.36 (m, 8H), 6.31 (1H, s), 3.97 (s, 2H), 3.87 (d, 2H), 2.77 (s, 4H), 2.37 (s, 3H), 1.77 (2H, m), 1.60 (2H, m).

Mass (m/z): 351.1 (M⁺+1).

### Example 11

### Synthesis of tartarate salt of 3-azabicyclo[3.1.0]hex-6-ylmethyl biphenyl-2-ylcarbamate (Compound No. 3)

Solid L (+) tartaric acid (0.65 mmol) was added to a solution of Compound No. 46 (200 mg, 0.65 mmol) in ethanol (10 mL) and the reaction mixture was refluxed for 1 hour. Solvent was evaporated under reduced pressure followed by the addition of diethyl ether. A sticky solid thus obtained was washed with diethylether and the supernatant was decanted. The residue was dried under high vacuum to furnish the title compound. Yield: 270 mg.

¹H NMR (CD₃OD) δ: 7.48-7.23 (9H, m), 4.44 (2H, s), 3.96-3.94 (2H, m), 3.49-3.32 (5H, m), 1.35-1.33 (3H, m).

### Example 12

### Synthesis of {3-[2-(1,3-benzodioxol-5-yl)ethyl]-3-azabicyclo[3.1.0]hex-6-yl}methyl (2-benzylphenyl)carbamate (Compound No. 31)

Potassium carbonate (94 mg) and potassium iodide (56.4 mg) were added to a solution of Compound No. 22 (0.12 g) and 5-(2-bromoethyl)-1,3-benzodioxole (78.5 mg) in acetonitrile (10.0 mL). The reaction mixture was refluxed for 2 hours. The solvent was concentrated under reduced pressure and the residue thus obtained was diluted with ethyl acetate and water. The ethyl acetate layer was concentrated under reduced pressure. The residue thus obtained was purified by column chromatography using ethyl acetate in hexane as eluent to furnish the title compound. Yield=143 mg.

¹H NMR (CDCl₃) δ: 7.30-7.11 (9H, m), 6.73-7.70 (3H, m), 6.35 (1H, s), 5.91 (2H, s), 3.97 (2H, s), 3.90-3.88 (2H, d), 3.16-2.15 (11H, m), 1.82-1.80 (4H, m).

### Example 13

### Synthesis of tert-butyl 8-[({[(4-fluorobenzyl)(phenyl)amino]carbonyl}oxy)methyl]-3-azabicyclo[3.2.1]octane-3-carboxylate (Compound No. 42)

### Step-I: Methyl (4-fluorobenzyl)phenyl carbamate

N,N-dimethyl aniline (1.09 g, 9.13 mmol) and ethyl chloroformate (971.8 mg, 8.96 mmol) were added to a solution of the compound N-(4-fluorobenzyl)aniline (1.8 g, 8.96 mmol) in tetrahydrofuran (30 mL). The reaction mixture was stirred in an ice-bath for 40 minutes, and then stirred at room temperature for 18 hours. The reaction mixture was concentrated under reduced pressure. The residue thus obtained was diluted with dichloromethane, washed with hydrochloric acid (1N), water and aqueous sodium bicarbonate solution and dried over anhydrous sodium sulphate. The organic layer was filtered and concentrated under reduced pressure. The residue thus obtained was purified by column chromatography using ethyl acetate in hexane as eluent to furnish the title compound. Yield: 1.9 g.

### Step-II: tert-butyl 8-[({[(4-fluorobenzyl)(phenyl)amino]carbonyl}oxy)methyl]-3-azabicyclo[3.2.1]octane-3-carboxylate

### Compound No. 42

Sodium hydride (21.11 mg, 0.88 mmol) was added to a solution of the compound obtained from step a above (200 mg, 0.733 mmol) and tert-butyl 8-(hydroxymethyl)-3-azabicyclo[3.2.1]octane-3-carboxylate (176.56 mg) in dry toluene (20 mL) and the reaction mixture was refluxed for 4 hours. The reaction mixture was concentrated under reduced pressure and the residue thus obtained was diluted with water and extracted with ethyl acetate. The organic layer was washed with water and brine, dried over anhydrous sodium sulphate, filtered and concentrated under reduced pressure. The residue thus obtained was purified by column chromatography using 12% ethyl acetate in hexane as eluent to furnish the title compound. Yield: 260 mg.

¹H NMR (CDCl₃) δ: 7.3-6.95 (m, 9H), 4.81 (s, 2H), 3.90 (d, 3H), 3.72 (d, 1H), 2.70 (dd, 2H), 1.95-1.85 (m, 3H), 1.62 (s, 2H), 1.44 (s, 11H)

Mass (m/z): 469.1 (M⁺+1).

Analogs of tert-butyl 8-[({[(4-fluorobenzyl)(phenyl)amino]carbonyl}oxy)methyl]-3-azabicyclo[3.2.1]octane-3-carboxylate (Compound No. 42) described below were prepared similarly using the appropriate corresponding reagents:

### (3-Benzyl-3azabicyclo[3.1.0]hex-6-yl)methyl benzyl (3-fluorophenyl)carbamate

### Compound No. 16

¹H NMR (CDCl₃) δ: 7.32-7.21 (m, 11H), 6.95-6.87 (m, 3H), 4.86 (s, 2H), 3.98 (d, 2H), 83.61 (s, 2H), 2.93 (2H, d), 2.32 (2H, d), 1.29-1.25 (s, 3H).

Mass (m/z): 431.1 (M⁺+1).

### (3-Benzyl-3azabicyclo[3.2.1]oct-8-yl)methyl benzyl (4-fluorophenyl)carbamate.

### Compound No. 24

¹H NMR (CDCl₃) δ: 6.93-7.30 (m, 14H), 4.81 (s, 2H), 3.92 (d, 2H), 3.45 (s, 2H), 2.65 (dd, 2H), 2.01 (d, 2H), 1.88 (s, 2H), 1.70 (m, 3H), 1.28 (s, 2H).

Mass (m/z): 459.1 (M⁺+1).

### (3-Benzyl-3-azabicyclo[3.2.1]oct-8-yl)methyl(2-fluorobenzyl)phenyl carbamate.

### Compound No. 25

¹H NMR (CDCl₃) δ: 7.32-6.95 (m, 14H), 4.94 (s, 2H), 3.92 (d, 2H), 3.45 (s, 2H), 2.64 (dd, 2H), 2.02 (d, 2H), 1.88 (s, 2H), 1.76-1.60 (m, 5H).

Mass (m/z): 459.1 (M⁺+1).

### (3-Benzyl-3-azabicyclo[3.2.1]oct-8-yl)methyl benzyl(phenyl) carbamate

### Compound No. 26

¹H NMR (CDCl₃) δ: 7.30-7.12 (m, 15H), 4.85 (s, 2H), 3.92 (d, 2H), 3.45 (s, 2H), 2.64 (dd, 2H), 2.01 (d, 2H), 1.88 (s, 2H), 1.76-1.60 (m, 5H).

Mass (m/z): 441 (M⁺+1).

### (3-Benzyl-3-azabicyclo[3.2.1]oct-8-yl)methyl(4-methylbenzyl)phenyl carbamate

### Compound No. 27

¹H NMR (CDCl₃) δ: 7.29-7.06 (m, 14H), 4.80 (s, 2H), 3.91 (d, 2H), 3.45 (s, 2H), 2.64 (dd, 2H), 2.30 (s, 3H), 2.01 (d, 2H), 1.88 (s, 2H), 1.76-1.60 (m, 5H).

Mass (m/z): 455 (M⁺+1).

### (3-Benzyl-3-azabicyclo[3.2.1]oct-8-yl)methyl(4-chlorobenzyl)phenylcarbamate

### Compound No. 32

¹H NMR (CDCl₃) δ: 7.30-7.18 (m, 12H), 7.05 (s, 2H), 4.82 (s, 2H), 3.93 (d, 2H), 3.46 (s, 2H), 2.69 (dd, 2H), 2.01 (dd, 2H), 1.89 (s, 2H), 1.73 (m, 3H), 1.60 (s, 2H).

Mass (m/z): 475.1 (M⁺+1).

### (3-Benzyl-3-azabicyclo[3.2.1]oct-8-yl)methyl benzyl(4-chlorophenyl)carbamate

### Compound No. 34

¹H NMR (CDCl₃) δ: 7.30-7.08 (m, 14H), 4.80 (s, 2H), 3.91 (d, 2H), 3.45 (s, 2H), 2.63 (dd, 2H), 2.01 (dd, 2H), 1.89 (s, 2H), 1.73 (m, 3H), 1.60 (s, 2H).

Mass (m/z): 475.1 (M⁺+1).

### (3-Benzyl-3-azabicyclo[3.2.1]oct-8-yl)methyl benzyl(3-fluorophenyl)carbamate

### Compound No. 36

¹H NMR (CDCl₃) δ: 7.31-7.20 (m, 11H), 6.90 (m, 3H), 4.85 (s, 2H), 3.94 (d, 2H), 3.45 (s, 2H), 2.64 (dd, 2H), 2.01 (dd, 2H), 1.89 (s, 2H), 1.73 (m, 3H), 1.60 (s, 2H).

Mass (m/z): 459.1 (M⁺+1).

### (3-Benzyl-3-azabicyclo[3.2.1]oct-8-yl)methyl cyclopentylmethyl)phenylcarbamate

### Compound No. 37

¹H NMR (CDCl₃) δ: 7.35-7.16 (m, 10H), 3.88 (d, 2H), 3.63 (d, 2H), 3.46 (s, 2H), 2.66 (d, 2H), 2.02 (d, 3H), 1.91 (s, 2H), 1.71-1.48 (m, 11H), 1.20 (m, 2H).

Mass (m/z): 433.2 (M⁺+1).

### (3-Benzyl-3-azabicyclo[3.2.1]oct-8-yl)methyl(3,5-difluorobenzyl)(3-fluorophenyl)carbamate

### Compound No. 38

¹H NMR (CDCl₃) δ: 7.26 (m, 6H), 6.93 (m, 3H), 6.72 (m, 3H), 4.81 (s, 2H), 3.94 (d, 2H), 3.45 (s, 2H), 2.64 (dd, 2H), 2.02 (dd, 2H), 1.89 (s, 2H), 1.73 (m, 3H), 1.60 (s, 2H).

Mass (m/z): 495.1 (M⁺+1).

### Tert-butyl 8-[({[(2-fluorobenzyl)(phenyl)amino]carbonyl}oxy)methyl]-3-azabicyclo[3.2.1]octane-3-carboxylate

### Compound No. 40

¹H NMR (CDCl₃) δ: 7.35-7.07 (m, 8H), 6.99 (m, 1H), 4.49 (s, 2H), 4.09 (m, 1H), 3.95 (m, 1H), 3.56 (d, 1H), 3.47 (d, 1H), 3.30 (2H, m), 1.43 (s, 11H), 0.95 (s, 1H)

Mass: 441.4 (M⁺+1).

### Tert-butyl 8-[({[(2-fluorobenzyl)(3-fluorophenyl)amino]carbonyl}oxy)methyl]-3-azabicyclo[3.2.1]octane-3-carboxylate

### Compound No. 44

¹H NMR (CDCl₃) δ: 7.30-6.92 (m, 8H), 4.94 (s, 2H), 3.92 (d, 2H), 3.86 (d, 1H), 3.72 (d, 1H), 2.74 (dd, 2H), 1.89 (m, 3H), 1.64 (s, 2H), 1.44 (s, 11H).

Mass: 487.1 (M⁺+1).

### (3-Benzyl-3-azabicyclo[3.2.1]oct-8-yl)methyl(3-hydroxy-4-methoxybenzyl)phenylcarbamate

### Compound No. 47

¹H NMR (CDCl₃) δ: 7.33-7.11 (m, 10H), 6.81 (s, 1H), 6.74 (d, 2H), 6.68 (d, 2H), 4.74 (s, 2H), 3.91 (d, 2H), 3.85 (s, 3H), 3.69 (s, 2H), 2.78 (d, 2H), 2.16 9d, 2H), 1.93 (s, 3H), 1.78 (s, 2H), 1.64 (s, 2H).

Mass: 487.2 (M⁺+1).

### (3-Benzyl-3-azabicyclo[3.2.1]oct-8-yl)methyl(1H-imidazol-4-ylmethyl)phenylcarbamate

### Compound No. 48

¹H NMR (CDCl₃) δ: 7.61 (s, 1H), 7.33-7.21 (m, 8H), 7.13 (s, 2H), 6.90 (s, 1H), 4.72 (s, 2H), 4.17 (d, 2H), 3.51 (s, 2H), 2.71 (d, 2H), 2.07 (d, 2H), 1.91 (s, 2H), 1.76 (s, 3H), 1.61 (s, 2H).

Mass: 431.1 (M⁺+1).

### (3-Benzyl-3-azabicyclo[3.2.1]oct-8-yl)methyl(4-tert-butylbenzyl)(3-fluorophenyl)carbamate

### Compound No. 49

¹H NMR (CDCl₃) δ: 7.31-7.21 (m, 8H), 7.13 (d, 2H), 6.98-6.87 (m, 3H), 4.82 (s, 2H), 3.92 (d, 2H), 3.47 (s, 2H), 2.65 (s, 2H), 2.04 (s, 2H), 1.86 (s, 3H), 1.75 (m, 2H), 1.60 (s, 2H), 1.29 (s, 9H).

Mass: 515.2 (M⁺+1).

### (3-Benzyl-3-azabicyclo[3.2.1]oct-8-yl)methyl(4-tert-butylbenzyl)phenylcarbamate

### Compound No. 50

¹H NMR (CDCl₃) δ: 7.39-7.06 (m, 14H), 4.82 (s, 2H), 3.91 (d, 2H), 3.45 (s, 2H), 2.62 (s, 2H), 2.00 (s, 2H), 1.86 (s, 2H), 1.74 (s, 3H), 1.59 (s, 2H), 1.29 (s, 9H)

Mass: 497.2 (M⁺+1).

### (3-Benzyl-3-azabicyclo[3.2.1]oct-8-yl)methyl(3,5-difluorobenzyl)phenylcarbamate

### Compound No. 51

¹H NMR (CDCl₃) δ: 7.39-7.12 (m, 10H), 6.77 (d, 2H), 6.69 (m, 1H), 4.81 (s, 2H), 3.93 (d, 2H), 3.49 (s, 2H), 2.68 (s, 2H), 2.06 (s, 2H), 1.90 (s, 2H), 1.74 (s, 3H), 1.61 (s, 2H).

Mass: 477.1 (M⁺+1).

### (3-Benzyl-3-azabicyclo[3.2.1]oct-8-yl)methyl(3,4-difluorobenzyl)(3-fluorophenyl)carbamate

### Compound No. 52

¹H NMR (CDCl₃): δ 7.30-7.23 (m, 6H), 7.09 (m, 2H), 6.92-6.82 (m, 4H), 4.78 (s, 2H), 3.94 (d, 2H), 3.47 (s, 2H), 2.67 (d, 2H), 2.03 (d, 2H), 1.90 (s, 2H), 1.74 (s, 3H), 1.61 (s, 2H)

Mass (m/z): 495.1 (M⁺+1)

### (3-Benzyl-3-azabicyclo[3.2.1]oct-8-yl)methyl(3,4-difluorobenzyl)phenylcarbamate

### Compound No. 53

¹H NMR (CDCl₃): δ 7.30-7.19 (m, 8H), 7.09-7.03 (m, 4H), 6.93 (s, 1H), 4.78 (s, 2H), 3.92 (d, 2H), 3.46 (s, 2H), 2.65 (d, 2H), 2.02 (d, 2H), 1.89 (s, 2H), 1.73 (s, 3H), 1.62 (d, 2H).

Mass (m/z): 477.1 (M⁺+1)

### (3-Benzyl-3-azabicyclo[3.2.1]oct-8-yl)methyl(3-fluorophenyl)[4-(trifluoromethyl)benzyl]carbamate

### Compound No. 54

¹H NMR (CDCl₃): δ 7.57 (d, 2H), 7.35-7.23 (m, 8H), 6.92 (m, 3H), 4.90 (s, 2H), 3.94 (s, 2H), 3.48 (s, 2H), 2.67 (s, 2H), 2.02 (s, 2H), 1.87 (s, 3H), 1.73 (s, 3H), 1.60 (s, 2H).

Mass (m/z): 527.1 (M⁺+1)

### (3-Benzyl-3-azabicyclo[3.2.1]oct-8-yl)methyl phenyl[4-(trifluoromethyl)benzyl]carbamate

### Compound No. 55

¹H NMR (CDCl₃): δ 7.56 (d, 2H), 7.35-7.21 (m, 10H), 7.11 (s, 2H), 4.90 (s, 2H), 3.92 (d, 2H), 3.46 (s, 2H), 2.66 (s, 2H), 2.01 (s, 2H), 1.87 (s, 2H), 1.72 (s, 3H), 1.60 (s, 2H)

Mass (m/z): 509.1 (M⁺+1)

### (3-Benzyl-3-azabicyclo[3.2.1]oct-8-yl)methyl(3-fluorophenyl)(4-hydroxybenzyl)carbamate

### Compound No. 56

¹H NMR (CDCl₃): δ 7.31-7.19 (m, 6H), 7.04 (d, 2H), 6.87 (m, 3H), 6.74 (d, 2H), 4.74 (s, 2H), 3.91 (d, 2H), 3.57 (s, 2H), 2.76 (d, 2H), 2.12 (d, 2H), 1.90 (s, 2H), 1.76 (m, 3H), 1.62 (s, 2H).

Mass (m/z): 475.1 (M⁺+1)

### (3-Benzyl-3-azabicyclo[3.2.1]oct-8-yl)methyl(3-fluorophenyl)(3-hydroxy-4-methoxybenzyl)carbamate

### Compound No. 57

¹H NMR (CDCl₃): δ 7.32-7.20 (m, 8H), 6.93-6.65 (m, 4H), 4.75 (s, 2H), 3.93 (d, 2H), 3.86 (s, 3H), 3.50 (s, 2H), 2.71 (s, 2H), 2.06 (s, 2H), 1.92 (s, 2H), 1.76 (s, 3H), 1.62 (s, 2H).

Mass (m/z): 505.1 (M⁺+1).

### 3-Benzyl-3-azabicyclo[3.2.1]oct-8-yl benzyl(2-fluorophenyl)carbamate

### Compound No. 68

¹H NMR (CDCl₃): δ 7.27-7.03 (14H, m), 4.83-4.77 (4H, bd), 3.15 (2H, bs), 2.41 (1H, bs), 2.25 (3H, bs), 2.08 (2H, bs), 1.94 (2H, bs), 1.74 (2H, bs).

Mass (m/z): 445 (M⁺+1).

### 3-benzyl-3-azabicyclo[3.2.1]oct-8-yl (2-fluorobenzyl)(3-fluorophenyl)carbamate

### Compound No. 70

¹H NMR (CDCl₃): δ 7.36-6.94 (13H, m), 4.97 (2H, s), 4.78 (1H, m), 3.22 (1H, s), 2.31 (2H, m), 2.12-2.03 (4H, bm), 1.78-1.64 (4H, m).

Mass (m/z): 463 (M⁺+1).

### 3-Benzyl-3-azabicyclo[3.2.1]oct-8-yl (4-methylbenzyl)phenylcarbamate

### Compound No. 71

¹H NMR (CDCl₃): δ 7.35-7.10 (14H, m), 4.83 (2H, s), 4.76 (1H, m), 3.19 (2H, s), 2.33-2.26 (5H, m), 2.10 (3H, bs), 1.76 (2H, d, 8 Hz), 1.64 (2H, bs).

Mass (m/z): 441 (M⁺+1).

### 3-Benzyl-3-azabicyclo[3.2.1]oct-8-yl (4-fluorobenzyl)phenylcarbamate

### Compound No. 72

¹H NMR (CDCl₃): δ 7.36-6.97 (14H, m), 4.83 (2H, s), 4.76 (1H, m), 3.20 (2H, bs), 2.34-2.27 (2H, bs), 2.10-2.05 (4H, bs), 1.77-1.64 (4H, m).

Mass (m/z): 445 (M⁺+1).

### 3-Benzyl-3-azabicyclo[3.2.1]oct-8-yl benzyl(4-fluorophenyl)carbamate

### Compound No. 73

¹H NMR (CDCl₃): δ 7.31-6.99 (14H, m), 4.83 (2H, s), 4.76 (1H, t, 4 Hz), 3.22 (2H, bs), 2.29 (2H, bs), 2.10 (3H, bs), 1.78-1.63 (4H, m).

Mass (m/z): 445 (M⁺+1).

### 3-Benzyl-3-azabicyclo[3.2.1]oct-8-yl benzyl(4-chlorophenyl)carbamate

### Compound No. 74

¹H NMR (CDCl₃): δ 7.32-7.09 (14H, m), 4.82 (2H, s), 4.76 (1H, m), 3.22 (1H, s), 2.31 (2H, bd), 2.11 (4H, bs), 1.78-1.64 (4H, m).

Mass (m/z): 461 (M⁺+1).

### 3-Benzyl-3-azabicyclo[3.2.1]oct-8-yl (4-chlorobenzyl)phenylcarbamate

### Compound No. 75

¹H NMR (CDCl₃): δ 7.36-7.13 (14H, m), 4.82 (2H, s), 4.74 (1H, m), 3.19 (2H, s), 2.28 (2H, bs), 2.09-2.04 (4H, bd), 1.76-1.62 (4H, m).

Mass (m/z): 461 (M⁺+1).

### Example 14

### Synthesis of hydrochloride salt of 3-azabicyclo[3.2.1]oct-8-ylmethyl (4-fluorobenzyl)phenyl carbamate (Compound No. 33)

### Step a (in-situ): 3-Azabicyclo[3.2.1]oct-8-ylmethyl (4-fluorobenzyl)phenyl carbamate

### Compound No. 43

Ethanolic hydrochloric acid was added to a solution of Compound No. 42 (260 mg) in ethanol (10 mL) and the reaction mixture was stirred for 2 hours to furnish the title compound in-situ.

### Step b: Hydrochloride salt of 3-azabicyclo[3.2.1]oct-8-ylmethyl (4-fluorobenzyl)phenyl carbamate

### Compound No. 33

The reaction mixture obtained from step a above was concentrated under reduced pressure to furnish the title compound. Yield: 52 mg.

¹H NMR (CDCl₃) δ: 9.88 (1H), 8.98 (1H), 7.32-6.95 (m, 9H), 4.79 (s, 2H), 3.89 (d, 2H), 3.16 (d, 2H), 2.91 (d, 2H), 2.08-1.97 (m, 5H), 1.86 (s, 2H).

Mass (m/z): 369.1 (M⁺+1).

Analogs of 3-azabicyclo[3.2.1]oct-8-ylmethyl (4-fluorobenzyl)phenyl carbamate (Compound No. 43) (in-situ) and hydrochloride salt of 3-azabicyclo[3.2.1]oct-8-ylmethyl (4-fluorobenzyl)phenyl carbamate (Compound No. 33) described below were prepared similarly using appropriate corresponding reagents:

### Hydrochloride salt of 3-azabicyclo[3.2.1]oct-8-ylmethyl (2-fluorobenzyl)(3-fluorophenyl)carbamate

### Compound No. 35

¹H NMR (CDCl₃) δ: 9.89 (b, 1H), 8.89 (b, 1H), 7.29-6.91 (m, 8H), 4.92 (s, 2H), 3.92 (d, 2H), 3.18 (d, 2H), 2.92 (d, 2H), 2.09-1.97 (m, 5H), 1.86 (s, 2H).

### 3-Azabicyclo[3.2.1]oct-8-ylmethyl (2-fluorobenzyl)(3-fluorophenyl)carbamate

### Compound No. 45

The title compound was prepared in-situ.

### Hydrochloride salt of (3-azabicyclo[3.2.1]oct-8-yl)methyl(2-benzoylphenyl)carbamate

### Compound No. 66

¹H NMR (CDCl₃) δ: 8.39 (1H, d, 8 Hz), 7.70 (2H, d, 8 Hz), 7.62-7.47 (5H, m), 7.06 (1H, t, 8 Hz), 3.98 (2H, bs), 3.27 (2H, bs), 3.05 (2H, bs), 2.37 (2H, bs), 2.16 (3H, bs), 1.99 (2H, bs).

Mass (m/z): 365 (M⁺+1).

### Hydrochloride salt of 3-azabicyclo[3.2.1]oct-8-ylmethyl phenyl[3-(trifluoromethyl)benzyl]carbamate

### Compound No. 69

¹H NMR (CDCl₃) δ: 7.56 (2H, d, 8 Hz), 7.35-7.06 (7H, m), 4.88 (2H, s), 3.90 (2H, d, 8 Hz), 3.16 (2H, bd), 2.91 (2H, bs), 2.08-1.86 (7H, m).

Mass (m/z): 365 (M⁺+1).

### Example 15

### Synthesis of 3-azabicyclo[3.1.0]hex-6-ylmethyl (2-fluorobenzyl)phenylcarbamate (Compound No. 14)

Ethanolic hydrochloric acid (10 mL) was added to a solution of Compound No. 40 (200 mg) in methanol (5 mL) and the reaction mixture was stirred for 2 hours at room temperature. The reaction mixture was concentrated and the residue thus obtained was diluted with water. The reaction mixture was basified with dilute potassium hydroxide and extracted with ethyl acetate. The organic layer was washed with water and brine, dried over anhydrous sodium sulphate, filtered and concentrated under reduced pressure. The residue thus obtained was purified by preparative column chromatography to furnish the title compound. Yield: 60 mg.

¹H NMR (CDCl₃) δ: 7.32-7.07 (8H, m), 4.93 (s, 2H), 4.06 (2H, d), 3.19 (2H, d), 3.09 (2H, d), 1.49 (2H, s), 1.19 (s, 1H).

Mass (m/z): 341.1 (M⁺+1).

### Example 16

### Synthesis of N-[(3-benzyl-3-azabicyclo[3.2.1]oct-8-yl)methyl]biphenyl-2-carboxamide (Compound No. 21)

Hydroxybenzotriazole (135 mg) and N-methylmorpholine (204 mg) were added to a solution of hydroxy(diphenyl)acetic acid (200 mg) and 1-(3-benzyl-3-azabicyclo[3.2.1]oct-8-yl)methanamine (230 mg) in dimethylformamide (10 mL) at 0° C. The resulting reaction mixture was stirred at 0° C. for one hour followed by the addition of 1-(3-dimethylaminopropyl)-3-ethylcarbodiimide hydrochloride (191.7 mg). The reaction mixture was further stirred at the same temperature for one hour and then at room temperature overnight. The reaction mixture was then quenched with sodium bicarbonate solution and then extracted with ethyl acetate. The ethyl acetate layer was washed with water and brine, dried over anhydrous sodium sulphate and concentrated under reduced pressure. The residue thus obtained was purified by column chromatography using ethyl acetate in hexane as eluent to furnish the title compound. Yield: 308 mg.

¹H NMR (CDCl₃) δ: 7.73-7.71 (1H, m), 7.47-7.30 (13H, m), 5.18 (1H, bs), 3.43 (2H, s), 2.98-2.94 (2H, m), 2.57-2.55 (2H, m), 1.89-1.87 (2H, m), 1.64-1.61 (3H, m), 1.56-1.49 (4H, m).

Analogs of N-[(3-benzyl-3-azabicyclo[3.2.1]oct-8-yl)methyl]biphenyl-2-carboxamide (Compound No. 21) were prepared similarly using appropriate corresponding reagents:

### 2-Benzyl-N-[(3-benzyl-3-azabicyclo[3.2.1]oct-8-yl)methyl]benzamide

### Compound No. 23

¹H NMR (CDCl₃) δ: 7.39-7.13 (14H, m), 5.57 (1H, s), 4.18 (2H, s), 3.46 (2H, s), 3.14-3.11 (2H, m), 2.67-2.63 (2H, m), 2.00-1.97 (2H, m), 1.84-1.69 (3H, m), 1.64-1.62 (4H, m).

### N-[(3-benzyl-3-azabicyclo[3.2.1]oct-8-yl)methyl]-2-phenoxybenzamide

### Compound No. 76

¹H NMR (CDCl₃) δ: 7.73 (2H, d, 8 Hz), 7.39-6.99 (12H, m), 6.06 (1H, bs), 3.54 (2H, bs), 3.27 (2H, t, 8 Hz), 2.77 (2H, bd), 2.15 (2H, bd), 2.05 (3H, bs), 1.90-1.75 (4H, bm).

Mass (m/z): 427 (M⁺+1).

### N-[(3-Benzyl-3-azabicyclo[3.2.1]oct-8-yl)methyl]-4-phenoxybenzamide

### Compound No. 77

¹H NMR (CDCl₃) δ: 8.23 (1H, d, 8 Hz), 7.64 (1H, bs), 7.38-7.1 (10H, m), 6.99 (2H, d, 8.8 Hz), 6.87 (1H, d, 8 Hz), 3.45 (2H, bs), 3.32-3.19 (2H, m), 2.63 (2H, bs), 1.97-1.90 (5H, bm), 1.65 (4H, bs).

Mass (m/z): 427 (M⁺+1).

### N-[(3-Benzyl-3-azabicyclo[3.2.1]oct-8-yl)methyl]-2-biphenyl-4-yl-N-methylacetamide

### Compound No. 78

¹H NMR (CDCl₃) δ: 7.58-7.53 (4H, m), 7.43 (2H, t, 8 Hz), 7.35-7.22 (8H, m), 3.75 (2H, s), 3.47 (2H, s), 3.26 (1H, d, 8 Hz), 3.13 (1H, d, 8 Hz), 2.99 (3H, d, 12 Hz), 2.68 (2H, bs), 2.07-2.03 (2H, bs), 1.96-1.90 (2H, m), 1.80-1.60 (5H, m).

Mass (m/z): 439 (M⁺+1).

### N-[(3-Benzyl-3-azabicyclo[3.2.1]oct-8-yl)methyl]-4-cyclohexyl-N-methylbenzamide

### Compound No. 79

¹H NMR (CDCl₃) δ: 7.30-7.19 (9H, bm), 3.49-3.39 (3H, bm), 3.16-2.98 (3H, m), 2.73-2.64 (2H, bd), 2.51 (1H, bs), 2.10 (1H, bs), 2.02 (2H, bs), 1.93-1.69 (10H, bs), 1.45-1.25 (6H, bm).

Mass (m/z): 431 (M⁺+1).

### N-[(3-benzyl-3-azabicyclo[3.2.1]oct-8-yl)methyl]-4-cyclohexylbenzamide

### Compound No. 80

¹H NMR (CDCl₃, 400 MHz): δ 7.68 (2H, d, 8 Hz), 7.32-7.25 (7H, m), 6.08 (1H, bs), 3.45 (2H, bs), 3.28-3.21 (2H, m), 2.73 (2H, bs), 2.54 (2H, bs), 2.04 (4H, bs), 1.86-1.25 (14H, m).

Mass (m/z): 417 (M⁺+1).

### N-[(3-benzyl-3-azabicyclo[3.2.1]oct-8-yl)methyl]-N-methylbiphenyl-4-carboxamide

### Compound No. 81

¹H NMR (CDCl₃) δ: 7.62-7.22 (14H, m), 3.50-3.41 (3H, bm), 3.20 (1H, bs), 3.13-3.02 (3H, bd), 2.74-2.66 (2H, m), 2.14-1.50 (9H, m).

Mass (m/z): 425 (M⁺+1).

### N-[(3-Benzyl-3-azabicyclo[3.2.1]oct-8-yl)methyl]-4′-(trifluoromethyl)biphenyl-2-carboxamide

### Compound No. 82

¹H NMR (CDCl₃) δ: 7.68-7.26 (13H, m), 5.23 (1H, bs), 3.44 (2H, bs), 3.05-2.98 (2H, m), 2.59 (2H, bs), 2.19 (2H, bs), 1.88-1.66 (7H, bs).

Mass (m/z): 479 (M⁺+1).

### N-[(3-Benzyl-3-azabicyclo[3.2.1]oct-8-yl)methyl]-N-methylbiphenyl-2-carboxamide

### Compound No. 83

¹H NMR (CDCl₃) δ: 7.66-7.21 (13H, m), 3.42 (2H, d), 2.84 (2H, s), 2.57 (2H, bs), 2.49 (3H, s), 1.90 (2H, d, 10 Hz), 1.66-1.45 (7H, m).

Mass (m/z): 493 (M⁺+1).

### N-[(3-Benzyl-3-azabicyclo[3.2.1]oct-8-yl)methyl]-2-biphenyl-4-ylacetamide

### Compound No. 84

¹H NMR (CDCl₃) δ: 7.58 (4H, d, 8 Hz), 7.45 (2H, m), 7.38-7.21 (8H, m), 5.40 (1H, bs), 3.60 (2H, s), 3.47 (2H, bs), 3.06 (2H, m), 2.68 (2H, bs), 2.03 (2H, bs), 1.88 (2H, bs), 1.42-1.25 (5H, bs).

Mass (m/z): 425 (M⁺+1).

### N-[(3-Benzyl-3-azabicyclo[3.2.1]oct-8-yl)methyl]-4-cyclohexyl-N-methylbenzamide

### Compound No. 85

¹H NMR (CDCl₃) δ: 7.30-7.19 (9H, m), 3.49-2.7 (6H, m), 2.73-2.51 (3H, m), 2.10 (1H, bs), 2.02 (2H, bs), 1.85-1.69 (10H, bm), 1.45-1.25 (6H, m).

Mass (m/z): 431 (M⁺+1).

### N-[(3-Benzyl-3-azabicyclo[3.2.1]oct-8-yl)methyl]biphenyl-4-carboxamide

### Compound No. 86

¹H NMR (CDCl₃) δ: 7.84 (2H, d, 8 Hz), 7.65 (2H, d, 8 Hz), 7.61 (2H, d, 7.2 Hz), 7.48-7.22 (8H, m), 6.14 (1H, bs), 3.49 (2H, s), 3.31 (2H, m), 2.72 (2H, m), 2.11-2.05 (4H, m), 1.78 (5H, bs).

Mass (m/z): 411 (M⁺+1).

### Example 17

### Synthesis of N-(3-azabicyclo[3.2.1]oct-8-ylmethyl)biphenyl-2-carboxamide (Compound No. 30)

The title compound was prepared following the procedure as described in Example 2, by deprotecting Compound No. 21 in place of Compound No. 1. In particular, palladium on carbon (10%) and ammonium formate were added to a solution of Compound No. 21 in methanol (35 mL). The reaction mixture was then refluxed for 1 hour and allowed to come to room temperature. The reaction mixture was filtered through a celite pad and washed with methanol. The filtrate was concentrated under reduced pressure and the residue thus obtained was dissolved in dichloromethane followed by the addition of water. The reaction mixture was basified with aqueous sodium hydroxide solution (10%). The organic layer was washed with water and brine, dried over anhydrous sodium sulphate and concentrated under reduced pressure to furnish the title compound.

¹H NMR (CDCl₃) δ: 7.73-7.70 (1H, m), 7.50-7.34 (8H, m), 5.22 (1H, s), 2.98-2.95 (2H, m), 2.88-2.85 (2H, m), 2.77-2.73 (2H, m), 1.81-1.72 (3H, m), 1.69-1.54 (4H, m).

Following compounds were prepared similarly using appropriate corresponding reagents:

### N-(3-Azabicyclo[3.2.1]oct-8-ylmethyl)-N-methyl-4′-(trifluoromethyl)biphenyl-2-carboxamide

### Compound No. 87

¹H NMR (CDCl₃) δ: 7.66-7.36 (8H, m), 5.30 (1H, s), 2.85-2.65 (6H, m), 2.50 (3H, s), 2.50-1.25 (8H, m).

Mass (m/z): 403 (M⁺+1).

### N-(3-Azabicyclo[3.2.1]oct-8-ylmethyl)-4-cyclohexylbenzamide

### Compound No. 88

¹H NMR (CDCl₃) δ: 7.69 (2H, d, 8 Hz), 7.27 (2H, d, 8 Hz), 6.12 (1H, bs), 3.28-3.23 (2H, m), 2.76 (3H, s), 2.5 (1H, bs), 2.01 (2H, bs), 1.93-0.96 (16H, m).

Mass (m/z): 327 (M⁺+1).

### N-(3-Azabicyclo[3.2.1]oct-8-ylmethyl)-4′-(trifluoromethyl)biphenyl-4-carboxamide

### Compound No. 89

¹H NMR (CDCl₃) δ: 7.69-7.35 (8H, m), 5.26 (1H, bs), 3.00-2.96 (2H, m), 2.66-2.54 (4H, m), 1.89-1.26 (7H, m).

Mass (m/z): 389 (M⁺+1).

### N-(3-Azabicyclo[3.2.1]oct-8-ylmethyl)biphenyl-4-carboxamide

### Compound No. 90

¹H NMR (CDCl₃) δ: 7.85 (2H, d, 8 Hz), 7.67 (2H, d, 8 Hz), 7.61 (2H, d, 8 Hz), 7.45 (2H, t, 8 Hz), 7.39 (1H, m), 6.24 (1H, bs), 3.55-3.28 (3H, m), 2.81 (3H, s), 2.06-1.13 (7H, m).

Mass (m/z): 321 (M⁺+1).

### N-(3-Azabicyclo[3.2.1]oct-8-ylmethyl)-2-biphenyl-4-yl-N-methylacetamide

### Compound No. 91

¹H NMR (CDCl₃) δ: 7.59-7.32 (9H, m), 3.76 (2H, s), 3.25-3.09 (2H, m), 3.03 (3H, m), 2.75 (4H, bs), 1.98-1.25 (7H, m).

Mass (m/z): 349.4 (M⁺+1).

### N-(3-Azabicyclo[3.2.1]oct-8-ylmethyl)-2-phenoxybenzamide

### Compound No. 92 (Compound No. 92)

¹H NMR (CDCl₃) δ: 8.24 (7H, m), 7.7 (1H, bs), 7.41-7.36 (3H, m), 7.27-7.18 (2H, m), 7.0 (2H, m), 6.89 (1H, m), 3.25-3.22 (2H, m), 2.77-2.64 (4H, m), 1.90-1.25 (7H, m).

Mass (m/z): 337 (M⁺+1).

### N-(3-Azabicyclo[3.2.1]oct-8-ylmethyl)-3-benzyl-N-methylbenzamide

### Compound No. 93

¹H NMR (CDCl₃) δ: 7.35-7.10 (9H, m), 4.04-4.00 (2H, bm), 3.08-2.87 (4H, m), 2.45 (3H, s), 2.06-1.25 (9H, m).

Mass (m/z): 349 (M⁺+1).

### Example 18

### Synthesis of (3-benzyl-3-azabicyclo[3.1.0]hex-6-yl)methyl benzyl(phenyl) carbamate (Compound No. 41)

### Step-I: Synthesis of N-benzylaniline

Sodium triacetoxyborohydride (5.97 g, 28.2 mmol) was added to the solution of benzaldehyde (1 g, 9.4 mmol) and aniline (960 mg, 10.34 mmol) in dichloroethane (30 mL) under nitrogen atmosphere. The reaction mixture was stirred at room temperature for 18 hours. The reaction mixture was concentrated under reduced and partitioned between ethyl acetate and 5% aqueous potassium hydroxide solution. The organic layer was washed with water and brine, dried over anhydrous sodium sulphate and concentrated under reduced pressure. The residue was purified by column chromatography using 2% ethyl acetate in hexane as eluent to furnish the title compound. Yield: 1.37 g.

### Step-II: Synthesis of 3-benzyl-3-azabicyclo[3.1.0]hex-6-yl methyl 1H-imidazole-1-carboxylate

(3-benzyl-3-azabicyclo[3.1.0]hex-6-yl)methanol (500 mg, 2.46 mmol was taken in dichloromethane (10 mL) and carbonyldiimidazole (599 mg, 3.69 mmol) was then added under nitrogen atmosphere and was stirred at room temperature for 4 hours. Water was added to the reaction mixture and then extracted with ethyl acetate. The organic layer was washed with water and brine, dried over anhydrous sodium sulphate and concentrated under reduced pressure to furnish the title compound.

### Step-III: Synthesis of (3-benzyl-3-azabicyclo[3.1.0]hex-6-yl)methyl benzyl (phenyl)carbamate

### Compound No. 41

Step-II intermediate (650 mg, 2.19 mmol) was taken in dry tetrahydrofuran (10 mL) and this solution was added to a solution of step-I intermediate (400 mg, 2.19 mmol) and n-butyllithium (1.37 mL, 2.19 mmol) in tetrahydrofuran at −10° C. The resulting reaction mixture was stirred for 2 hours at the same temperature, subsequently at room temperature overnight and then allowed to stand at room temperature. The reaction mixture was quenched with saturated ammonium chloride and extracted with ethyl acetate. The organic layer was washed with water and brine, dried over anhydrous sodium sulphate and concentrated under reduced pressure. The residue thus obtained was purified by column chromatography using 11% ethyl acetate in hexane as eluent to furnish the title compound. Yield: 550 mg.

¹H NMR (CDCl₃) δ: 7.33-7.13 (15H, m), 5.30 (s, 2H), 3.96 (d, 2H), 3.51 (s, 2H), 2.93 (d, 2H), 2.32 (d, 2H), 1.15-1.29 (m, 3H).

Mass (m/z): 413.1 (M⁺+1).

### Example 19

### Synthesis of 3-azabicyclo[3.1.0]hex-6-ylmethyl benzyl(phenyl)carbamate (Compound No. 15)

Ammonium formate (443 mg, 7.03 mmol) and palladium on carbon (50 mg, 10%) were added to a solution of Compound No. 41 (500 mg, 1.21 mmol) in methanol (10 mL) and refluxed for 1 hour. The reaction mixture was cooled to room temperature, filtered through a celite pad and washed with methanol. The filtrate was concentrated under reduced pressure. The residue thus obtained was diluted with water and acidified with hydrochloric acid. Impurities were extracted with diethylether. The aqueous layer was basified and extracted with ethyl acetate. Organic layer was separated, washed with water and brine, dried, filtered and concentrated under reduced pressure to furnish the title compound. Yield: 310 mg.

¹H NMR (CDCl₃) δ: 7.32-7.19 (m, 8H), 7.115 (s, 2H), 4.85 (s, 2H), 4.05 (2H, d), 3.30 (2H, d), 3.23 (d, 2H), 1.56 (s, 2H), 1.356 (s, 1H).

Mass (m/z): 323.1 (M⁺+1).

### Example 20

### Biological Activity

### Radioligand Binding Assays:

The affinity of test compounds for M₂ and M₃ muscarinic receptor subtypes was determined by [³H]-N-Methylscopolamine (NMS) binding studies using rat heart and submandibular gland respectively as described by Moriya et al., (Life Sci, 1999, 64 (25): 2351-2358) with minor modifications. Specific binding of [³H]-NMS was also determined using membranes from Chinese hamster ovary (CHO) cells expressing cloned human muscarinic receptor subtypes.

### Membrane Preparation:

### (a) Rat Tissues

Submandibular glands and heart were isolated and placed in ice-cold homogenizing buffer (HEPES 20 mM, 10 mM EDTA, pH 7.4) immediately after sacrifice. The tissues were homogenized in ten volumes of homogenizing buffer and the homogenate was filtered through two layers of wet gauze and filtrate was centrifuged at 500 g for 10 min. The supernatant was subsequently centrifuged at 40,000 g for 20 min. The pellet thus obtained was resuspended in assay buffer (HEPES 20 mM, EDTA 5 mM, pH 7.4) and were stored at −70° C. until the time of assay.

### (b) CHO Cells Expressing Human Recombinant Receptors

The cell pellets were homogenized for 30 seconds at 12,000 to 14,000 rpm, with intermittent gaps of 10-15 seconds in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer containing 10% sucrose and was stored at −70° C. until the time of assay.

### Ligand Binding Assay:

The compounds were dissolved and diluted in dimethyl sulphoxide. The membrane homogenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fiber filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μL of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki was calculated from competitive binding studies by using Cheng & Prusoff's equation (Biochem Pharmacol., 1973, 22: 3099-3108), Ki=IC₅₀/(1+[L]/Kd), where [L] is the concentration of ligand [³H]-N-methyl scopolamine used in the particular experiment and Kd is the estimate of affinity of receptors to the ligand.

Compounds described herein exhibited Ki values for M₂ receptors from about 4 nM to about 2170 nM, from about 4 nM to about 250 nM, from about 4 nM to about 32 nM and even from about 4 nM to about 17 nM.

Compounds described herein exhibited Ki values for M₃ receptors from about 0.1 nM to about 1000 nM, from about 0.1 nM to about 150 nM, from about 0.1 nM to about 55 nM and even from about 0.1 nM to about 12 nM.

We claim:

1. A compound of Formula I 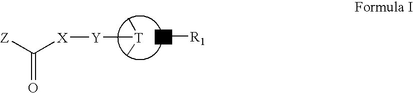
and pharmaceutically accepted salts, pharmaceutically acceptable solvates, enantiomers, diastereomers, polymorphs or N-oxides thereof, wherein 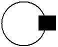
represents a nitrogen containing cyclic ring have 4-8 carbons;
T is a bridging group selected from —(CH₂)ₙ—, —CH(Q)CH₂—, —CH₂CH(Q)CH₂—, —CH(Q)—, —CH₂—O—CH₂— or —CH₂—NH—CH₂—,
wherein
the bridging group is attached to two carbon atoms of the ring 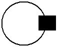
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 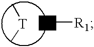
Z is —NHR₂, —N(R₂)₂, aryl or cycloalkyl,
wherein
R₂ is independently selected from alkyl, aryl, aralkyl, heteroaryl, cycloalkyl, heterocyclyl, heterocyclylalkyl or heteroarylalkyl; and
R₁ is selected from hydrogen, aralkyl or Rᵤ,
wherein
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring.

2. A compound selected from:
(3-Benzyl-3-azabicyclo[3.1.0]hex-6-yl)methyl biphenyl-2-ylcarbamate (Compound No. 1),
N-[(3-benzyl-3-azabicyclo[3.1.0]hex-6-yl)methyl]-N′-biphenyl-2-ylurea (Compound No. 2),
Tartarate salt of 3-azabicyclo[3.1.0]hex-6-ylmethyl biphenyl-2-ylcarbamate (Compound No. 3),
3-Benzyl-3-azabicyclo[3.2.1]oct-8-yl biphenyl-2-ylcarbamate (Compound No. 4),
3-azabicyclo[3.2.1]oct-8-yl biphenyl-2-ylcarbamate (Compound No. 5),
2-Benzyl-2-azabicyclo[2.2.1]hept-7-yl biphenyl-2-ylcarbamate (Compound No. 6),
(3-Benzyl-3-azabicyclo[3.2.1]oct-8-yl)methyl biphenyl-2-ylcarbamate (Compound No. 7),
(3-Benzyl-3-azabicyclo[3.1.0]hex-1-yl)methyl biphenyl-2-ylcarbamate (Compound No. 8),
(3-Benzyl-3-azabicyclo[3.1.0]hex-6-yl)methyl[2-(2-thienyl)phenyl]carbamate (Compound No. 9),
3-azabicyclo[3.1.0]hex-6-ylmethyl[2-(2-thienyl)phenyl]carbamate (Compound No. 10),
(3-Benzyl-3-azabicyclo[3.1.0]hex-6-yl)methyl(2′,4′-difluorobiphenyl-2-yl)carbamate (Compound No. 11),
(3-Benzyl-3-azabicyclo[3.1.0]hex-1-yl)methyl(2′,4′-difluorobiphenyl-2-yl)carbamate (Compound No. 12),
(3-Benzyl-3-azabicyclo[3.1.0]hex-6-yl)methyl(2′,4′-dimethoxybiphenyl-2-yl)carbamate (Compound No. 13),
3-Azabicyclo[3.1.0]hex-6-ylmethyl (2-fluorobenzyl)phenylcarbamate (Compound No. 14),
3-Azabicyclo[3.1.0]hex-6-ylmethyl benzyl(phenyl)carbamate (Compound No. 15),
(3-Benzyl-3-azabicyclo[3.1.0]hex-6-yl)methyl benzyl(3-fluorophenyl)carbamate (Compound No. 16),
3-Azabicyclo[3.1.0]hex-6-ylmethyl (2′,4′-difluorobiphenyl-2-yl)carbamate (Compound No. 17),
3-Azabicyclo[3.1.0]hex-6-ylmethyl (2′,4′-dimethoxybiphenyl-2-yl)carbamate (Compound No. 18),
3-Benzyl-3-azabicyclo[3.1.0]hex-6-yl)methyl[2-(1,3-benzodioxol-5-yl)phenyl]carbamate (Compound No. 19),
(3-Benzyl-3-azabicyclo[3.2.1]oct-8-yl)methyl(2-benzylphenyl)carbamate (Compound No. 20),
N-[(3-benzyl-3-azabicyclo[3.2.1]oct-8-yl)methyl]biphenyl-2-carboxamide (Compound No. 21),
3-Azabicyclo[3.1.0]hex-6-ylmethyl (2-benzylphenyl)carbamate (Compound No. 22),
2-Benzyl-N-[(3-benzyl-3-azabicyclo[3.2.1]oct-8-yl)methyl]benzamide (Compound No. 23),
(3-Benzyl-3-azabicyclo[3.2.1]oct-8-yl)methyl benzyl (4-fluorophenyl)carbamate. (Compound No. 24),
(3-Benzyl-3-azabicyclo[3.2.1]oct-8-yl)methyl(2-fluorobenzyl)phenyl carbamate. (Compound No. 25),
(3-Benzyl-3-azabicyclo[3.2.1]oct-8-yl)methyl benzyl(phenyl)carbamate (Compound No. 26),
(3-Benzyl-3-azabicyclo[3.2.1]oct-8-yl)methyl(4-methylbenzyl)phenyl carbamate (Compound No. 27),
(3-benzyl-3-azabicyclo[3.2.1]oct-8-yl)methyl(2-benzoylphenyl)carbamate (Compound No. 28)
(3-Benzyl-3-azabicyclo[3.2.1]oct-8-yl)methyl[2-(4-methylbenzyl)phenyl]carbamate (Compound No. 29),
N-(3-azabicyclo[3.2.1]oct-8-ylmethyl)biphenyl-2-carboxamide (Compound No. 30),
{3-[2-(1,3-Benzodioxol-5-yl)ethyl]-3-azabicyclo[3.2.1]oct-8-yl}methyl (2-benzylphenyl)carbamate (Compound No. 31),
(3-Benzyl-3-azabicyclo[3.2.1]oct-8-yl)methyl(4-chlorobenzyl)phenylcarbamate (Compound No. 32),
3-Azabicyclo[3.2.1]oct-8-ylmethyl (4-fluorobenzyl)phenylcarbamate (Compound No. 33),
(3-Benzyl-3-azabicyclo[3.2.1]oct-8-yl)methyl benzyl(4-chlorophenyl)carbamate (Compound No. 34),
Hydrochloride salt of 3-azabicyclo[3.2.1]oct-8-ylmethyl (2-fluorobenzyl)(3-fluorophenyl)carbamate (Compound No. 35),
(3-Benzyl-3-azabicyclo[3.2.1]oct-8-yl)methyl benzyl(3-fluorophenyl)carbamate (Compound No. 36),
(3-Benzyl-3-azabicyclo[3.2.1]oct-8-yl)methyl(cyclopentylmethyl)phenylcarbamate (Compound No. 37),
(3-Benzyl-3-azabicyclo[3.2.1]oct-8-yl)methyl(3,5-difluorobenzyl)(3-fluorophenyl)carbamate (Compound No. 38),
(3-Benzyl-3-azabicyclo[3.1.0]hex-1-yl)methyl[2-(2-thienyl)phenyl]carbamate (Compound No. 39),
Tert-butyl 6-[({[(2-fluorobenzyl)(phenyl)amino]carbonyl}oxy)methyl]-3-azabicyclo[3.1.0]hexane-3-carboxylate (Compound No. 40),
(3-Benzyl-3-azabicyclo[3.1.0]hex-6-yl)methyl benzyl(phenyl)carbamate (Compound No. 41),
Tert-butyl 8-[({[(4-fluorobenzyl)(phenyl)amino]carbonyl}oxy)methyl]-3-azabicyclo[3.2.1]octane-3-carboxylate (Compound No. 42),
3-Azabicyclo[3.2.1]oct-8-ylmethyl (4-fluorobenzyl)phenylcarbamate (Compound No. 43),
Tert-butyl 8-[({[(2-fluorobenzyl)(3-fluorophenyl)amino]carbonyl}oxy)methyl]-3-azabicyclo[3.2.1]octane-3-carboxylate (Compound No. 44),
3-Azabicyclo[3.2.1]oct-8-ylmethyl (2-fluorobenzyl)(3-fluorophenyl)carbamate (Compound No. 45),
3-Azabicyclo[3.1.0]hex-6-ylmethyl biphenyl-2-ylcarbamate (Compound No. 46),
(3-Benzyl-3-azabicyclo[3.2.1]oct-8-yl)methyl(3-hydroxy-4-methoxyphenyl)phenylcarbamate (Compound No. 47),
(3-Benzyl-3-azabicyclo[3.2.1]oct-8-yl)methyl 1H-imidazol-4-yl(phenyl)carbamate (Compound No. 48),
(3-Benzyl-3-azabicyclo[3.2.1]oct-8-yl)methyl(4-tert-butylphenyl)(3-fluorophenyl)carbamate (Compound No. 49),
(3-Benzyl-3-azabicyclo[3.2.1]oct-8-yl)methyl(4-tert-butylphenyl)phenylcarbamate (Compound No. 50),
(3-Benzyl-3-azabicyclo[3.2.1]oct-8-yl)methyl(3,5-difluorophenyl)phenylcarbamate (Compound No. 51),
(3-Benzyl-3-azabicyclo[3.2.1]oct-8-yl)methyl(3,4-difluorophenyl)(3-fluorophenyl)carbamate (Compound No. 52),
(3-Benzyl-3-azabicyclo[3.2.1]oct-8-yl)methyl(3,4-difluorophenyl)phenylcarbamate (Compound No. 53),
(3-Benzyl-3-azabicyclo[3.2.1]oct-8-yl)methyl(3-fluorophenyl)[4-(trifluoromethyl)phenyl]carbamate (Compound No. 54),
(3-Benzyl-3-azabicyclo[3.2.1]oct-8-yl)methyl phenyl[4-(trifluoromethyl)phenyl]carbamate (Compound No. 55),
(3-Benzyl-3-azabicyclo[3.2.1]oct-8-yl)methyl(3-fluorophenyl)(4-hydroxyphenyl)carbamate (Compound No. 56),
(3-Benzyl-3-azabicyclo[3.2.1]oct-8-yl)methyl(3-fluorophenyl)(3-hydroxy-4-methoxyphenyl)carbamate (Compound No. 57),
(3-Benzyl-3-azabicyclo[3.2.1]oct-8-yl)methyl(2-ethoxyphenyl)carbamate (Compound No. 58),
(3-Benzyl-3-azabicyclo[3.2.1]oct-8-yl)methyl(2-hydroxy-3-methoxyphenyl)carbamate (Compound No. 59),
(3-Benzyl-3-azabicyclo[3.2.1]oct-8-yl)methyl(3,4-dimethoxyphenyl)carbamate (Compound NO. 60),
(3-Benzyl-3-azabicyclo[3.2.1]oct-8-yl)methyl biphenyl-2-ylcarbamate (Compound No. 61),
(3-Benzyl-3-azabicyclo[3.2.1]oct-8-yl)methyl(4-phenoxyphenyl)carbamate (Compound No. 62),
(3-Benzyl-3-azabicyclo[3.2.1]oct-8-yl)methyl biphenyl-4-ylcarbamate (Compound No. 63),
(3-Benzyl-3-azabicyclo[3.2.1]oct-8-yl)methyl[2-(4-methoxybenzyl)phenyl]carbamate (Compound No. 64),
(3-Benzyl-3-azabicyclo[3.2.1]oct-8-yl)methyl[2-(3-methoxybenzoyl)phenyl]carbamate (Compound No. 65),
Hydrochloride salt of 3-azabicyclo[3.2.1]oct-8-yl)methyl(2-benzoylphenyl)carbamate (Compound No. 66),
(3-Benzyl-3-azabicyclo[3.2.1]oct-8-yl)methyl[2-(4-methylbenzoyl)phenyl]carbamate (Compound No. 67),
3-Benzyl-3-azabicyclo[3.2.1]oct-8-yl benzyl(2-fluorophenyl)carbamate (Compound No. 68),
Hydrochloride salt of 3-azabicyclo[3.2.1]oct-8-ylmethyl phenyl[3-(trifluoromethyl)benzyl]carbamate (Compound No. 69),
3-benzyl-3-azabicyclo[3.2.1]oct-8-yl (2-fluorobenzyl)(3-fluorophenyl)carbamate (Compound No. 70),
3-Benzyl-3-azabicyclo[3.2.1]oct-8-yl (4-methylbenzyl)phenylcarbamate (Compound No. 71),
3-Benzyl-3-azabicyclo[3.2.1]oct-8-yl (4-fluorobenzyl)phenylcarbamate (Compound No. 72),
3-Benzyl-3-azabicyclo[3.2.1]oct-8-yl benzyl(4-fluorophenyl)carbamate (Compound No. 73),
3-Benzyl-3-azabicyclo[3.2.1]oct-8-yl benzyl(4-chlorophenyl)carbamate (Compound No. 74),
3-Benzyl-3-azabicyclo[3.2.1]oct-8-yl (4-chlorobenzyl)phenylcarbamate (Compound No. 75),
N-[(3-benzyl-3-azabicyclo[3.2.1]oct-8-yl)methyl]-2-phenoxybenzamide (Compound No. 76),
N-[(3-Benzyl-3-azabicyclo[3.2.1]oct-8-yl)methyl]-4-phenoxybenzamide (Compound No. 77),
N-[(3-Benzyl-3-azabicyclo[3.2.1]oct-8-yl)methyl]-2-biphenyl-4-yl-N-methylacetamide (Compound No. 78),
N-[(3-Benzyl-3-azabicyclo[3.2.1]oct-8-yl)methyl]-4-cyclohexyl-N-methylbenzamide (Compound No. 79),
N-[(3-benzyl-3-azabicyclo[3.2.1]oct-8-yl)methyl]-4-cyclohexylbenzamide (Compound No. 80),
N-[(3-benzyl-3-azabicyclo[3.2.1]oct-8-yl)methyl]-N-methylbiphenyl-4-carboxamide (Compound No. 81),
N-[(3-Benzyl-3-azabicyclo[3.2.1]oct-8-yl)methyl]-4′-(trifluoromethyl)biphenyl-2-carboxamide (Compound No. 82),
N-[(3-Benzyl-3-azabicyclo[3.2.1]oct-8-yl)methyl]-N-methylbiphenyl-2-carboxamide (Compound No. 83),
N-[(3-Benzyl-3-azabicyclo[3.2.1]oct-8-yl)methyl]-2-biphenyl-4-ylacetamide (Compound No. 84),
N-[(3-Benzyl-3-azabicyclo[3.2.1]oct-8-yl)methyl]-4-cyclohexyl-N-methylbenzamide (Compound No. 85),
N-[(3-Benzyl-3-azabicyclo[3.2.1]oct-8-yl)methyl]biphenyl-4-carboxamide (Compound No. 86),
N-(3-Azabicyclo[3.2.1]oct-8-ylmethyl)-N-methyl-4′-(trifluoromethyl)biphenyl-2-carboxamide (Compound No. 87),
N-(3-Azabicyclo[3.2.1]oct-8-ylmethyl)-4-cyclohexylbenzamide (Compound No. 88),
N-(3-Azabicyclo[3.2.1]oct-8-ylmethyl)-4′-(trifluoromethyl)biphenyl-4-carboxamide (Compound No. 89),
N-(3-Azabicyclo[3.2.1]oct-8-ylmethyl)biphenyl-4-carboxamide (Compound No. 90),
N-(3-Azabicyclo[3.2.1]oct-8-ylmethyl)-2-biphenyl-4-yl-N-methylacetamide (Compound No. 91),
N-(3-Azabicyclo[3.2.1]oct-8-ylmethyl)-2-phenoxybenzamide (Compound No. 92) or
N-(3-Azabicyclo[3.2.1]oct-8-ylmethyl)-3-benzyl-N-methylbenzamide (Compound No. 93).

3. A pharmaceutical composition comprising a therapeutically effective amount of a compound of Formula I and one or more pharmaceutically acceptable carriers, excipients or diluents, wherein the compound of Formula I is: 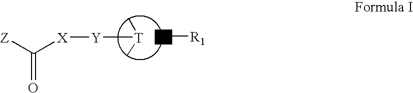
or a pharmaceutically accepted salt, pharmaceutically acceptable solvate, enantiomer, diastereomer, polymorph or N-oxides thereof, wherein 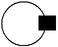
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 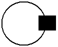
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 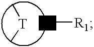
Z is —NHR₂, —N(R₂)₂, aryl or cycloalkyl,
wherein
R₂ is independently selected from alkyl, aryl, aralkyl, heteroaryl, cycloalkyl, heterocyclyl, heterocyclylalkyl or heteroarylalkyl; and
R₁ is selected from hydrogen, aralkyl or Rᵤ,
wherein
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring.

4. The pharmaceutical composition of claim 3 further comprising and one or more therapeutic ingredients selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or mixtures thereof.

5. A pharmaceutical composition comprising a therapeutically effective amount of a compound of claim 2 and one or more pharmaceutically acceptable carriers, excipients or diluents.

6. The pharmaceutical composition of claim 5 further comprising one or more therapeutic ingredients selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, PDE-IV inhibitors or mixtures thereof.

7. A method of treating or preventing a disease or disorder of the respiratory, urinary or gastrointestinal system, wherein the disease or disorder is mediated through muscarinic receptors in mammal comprising administering to a patient in need thereof a therapeutically effective amount of a compound of Formula I, wherein the compound of Formula I is: 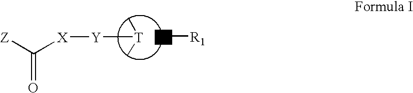
or a pharmaceutically accepted salt, pharmaceutically acceptable solvate, enantiomer, diastereomer, polymorph or N-oxides thereof, wherein 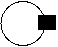
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 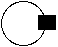
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 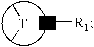
Z is —NHR₂, —N(R₂)₂, aryl or cycloalkyl,
wherein
R₂ is independently selected from alkyl, aryl, aralkyl, heteroaryl, cycloalkyl, heterocyclyl, heterocyclylalkyl or heteroarylalkyl; and
R₁ is selected from hydrogen, aralkyl or Rᵤ,
wherein
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring.

8. The method of claim 7, wherein the disease or disorder of the respiratory, urinary or gastrointestinal system is selected from urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

9. A method of treating or preventing a disease or disorder of the respiratory, urinary or gastrointestinal system, wherein the disease or disorder is mediated through muscarinic receptors in mammal comprising administering to a patient in need thereof a therapeutically effective amount of a compound of claim 2.

10. The method of claim 9, wherein the disease or disorder of the respiratory, urinary or gastrointestinal system is selected from urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

11. A method of preparing a compound of Formula VI or a compound of Formula V comprising the steps of: 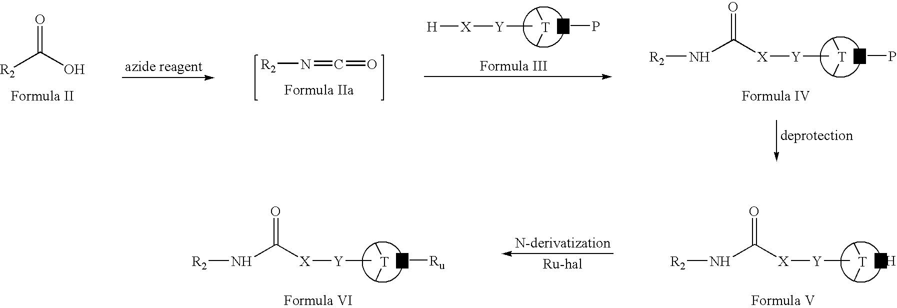
a) reacting a compound of Formula II with an azide reagent to form a compound of Formula IIa,
b) reacting the compound of Formula IIa with a compound of Formula III to form a compound of Formula IV,
c) deprotecting the compound of Formula IV to form a compound of Formula V, and
d) optionally N-derivatizing a compound of Formula V with a compound of
Formula Rᵤ-hal to form a compound of Formula VI,
wherein 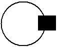
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 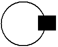
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom;
wherein when Y is no atom then X is directly attached to the ring 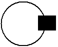
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring; and
R₂ is independently selected from alkyl, aryl, aralkyl, heteroaryl, cycloalkyl, heterocyclyl, heterocyclylalkyl or heteroarylalkyl.

12. A method of preparing a compound of Formula XI or a compound of Formula XIa comprising the steps of: 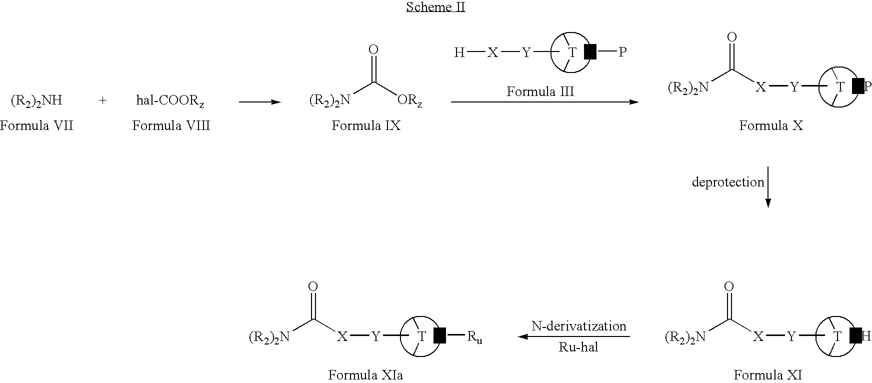
a) condensing a compound of Formula VII with compound of Formula VIII to form a compound of Formula IX,
b) reacting a compound of Formula IX with compound of Formula III to form a compound of Formula X,
c) deprotecting a compound of Formula X to form a compound of Formula XI, and
d) optionally N-derivatizing a compound of Formula XI with a compound of Formula Rᵤ-hal to form a compound of Formula XIa,
wherein 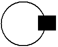
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 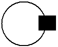
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 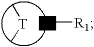
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring;
R₂ is independently selected from alkyl, aryl, aralkyl, heteroaryl, cycloalkyl, heterocyclyl, heterocyclylalkyl or heteroarylalkyl;
P is a protecting group selected from aralkyl, —C(═O)OC(CH₃)₃, —(═O)OC(CH₃)₂CHBr₂ or C(═O)OC(CH₃)₂CCl₃;
R_{z} is alkyl or aryl; and
hal is Br, Cl or I.

13. A method of preparing a compound of Formula XIII or a compound of Formula XIIIa comprising the steps of: 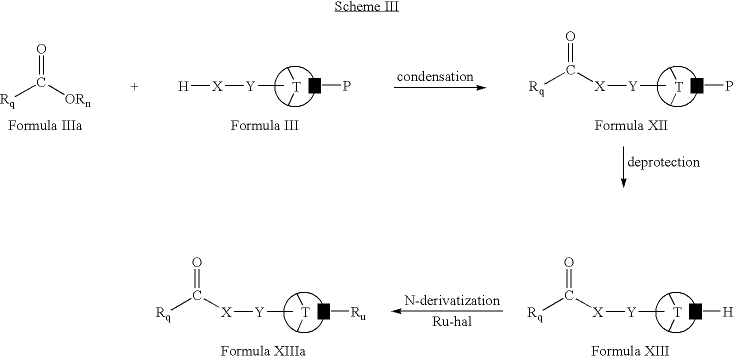
a) condensing a compound of Formula IIIa with a compound of Formula III to form a compound of Formula XII;
b) deprotecting a compound of Formula XII to form a compound of Formula XIII, and
c) optionally N-derivatizing a compound of Formula XIII with a compound of Formula Rᵤ-hal to form a compound of Formula XIIIa,
wherein 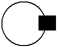
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 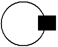
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 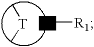
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring;
R₂ is independently selected from alkyl, aryl, aralkyl, heteroaryl, cycloalkyl, heterocyclyl, heterocyclylalkyl or heteroarylalkyl;
R_{q} is aryl or cycloalkyl;
Rₙ is hydrogen or alkyl;
P is a protecting group selected from aralkyl, —C(═O)OC(CH₃)₃, —C(═O)OC(CH₃)₂CHBr₂ or C(═O)OC(CH₃)₂CCl₃; and
hal is Br, Cl or I.

14. A method of preparing a compound of Formula XVII or a compound of Formula XVIII comprising the steps of: 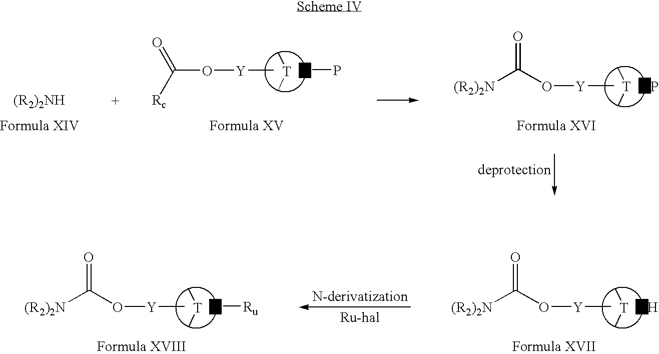
a) condensing a compound of Formula XIV with a compound of Formula XV to form a compound of Formula XVI;
b) deprotecting a compound of Formula XVI to form a compound of Formula XVII; and
c) N-derivatizing a compound of Formula XVII with a compound of Formula Ru-hal to form a compound of Formula XVIII,
wherein 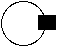
represents a nitrogen containing cyclic ring have 4-8 carbons;
T is a bridging group selected from —(CH₂)ₙ—, —CH(Q)CH₂—, —CH₂CH(Q)CH₂—, —CH(Q)-, —CH₂—O—CH₂— or —CH₂—NH—CH₂—,
wherein
the bridging group is attached to two carbon atoms of the ring 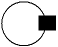
Q is alkyl, alkenyl, alkynyl, cycloalkyl, aryl, heteroaryl, heterocyclyl, aralkyl, heterocyclylalkyl or heteroarylalkyl; and
n is an integer selected from 0-3 (wherein when n is zero then T represents a direct bond);
X is O, S or NRₛ,
wherein
Rₛ is selected from hydrogen, alkyl, cycloalkyl, aryl, heteroaryl, heterocyclyl, heteroaryl, aralkyl, heteroarylalkyl or heterocyclylalkyl;
Y is alkylene or no atom,
wherein when Y is no atom then X is directly attached to the ring 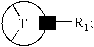
Rᵤ is alkyl, halogen, aryl, heteroaryl, cycloalkyl, heterocyclyl, heteroarylalkyl, heterocyclylalkyl, —C(═O)NRₓR_{y}, —COOR₂, —SO₂R₃, acyl,
wherein
R₃ is alkyl, aryl, heteroaryl, heterocyclyl, cycloalkyl, aralkyl, heteroarylalkyl, heterocyclylalkyl or —NRₓR_{y}, and
Rₓ and R_{y} are independently selected from hydrogen, alkyl, cycloalkyl, aryl, halogen, aralkyl, heteroaryl, heterocyclyl, heteroarylalkyl or heterocyclylalkyl; or Rₓ and R_{y} may also together join to form a heterocyclyl ring;
R₂ is independently selected from alkyl, aryl, aralkyl, heteroaryl, cycloalkyl, heterocyclyl, heterocyclylalkyl or heteroarylalkyl;
R_{q} is aryl or cycloalkyl;
Rₙ is hydrogen or alkyl;
P is a protecting group selected from aralkyl, —C(═O)OC(CH₃)₃, —C(═O)OC(CH₃)₂CHBr₂ or C(═O)OC(CH₃)₂CCl₃; and
R_{c} is heteroaryl or aryl.
